(19) United States
(12) Patent Application Publication
(10) Pub. No.: US 2011/0201665 A1
(43) Pub. Date: Aug. 18, 2011

(54) Compositions, Methods, and Kits for Treating Influenza Viral Infections

(75) Inventors: Ralf Altmeyer, Singapore (SG); Geeta Sharma, Singapore (SG); Vishal Vikas Pendharkar, Singapore (SG); Danilal Champalal Sharma, Singapore (SG)

(73) Assignees: COMBINATORX (SINGAPORE) PTE. LTD., Helios (SG); EXCRX (Singapore) PTE. LTD. (F.K.A. CombinatoRx (Singapore) PTE. LTD., Helios (SG)

(21) Appl. No.: 12/855,046

(22) Filed: Aug. 12, 2010

Related U.S. Application Data
(63) Continuation-in-part of application No. 12/708,076, filed on Feb. 18, 2010.
(60) Provisional application No. 61/153,418, filed on Feb. 18, 2009.

(30) Foreign Application Priority Data
Feb. 18, 2010 (IB) PCT/IB2010/000514

(51) Int. Cl. A61K 31/4015; A61P 31/16

(52) U.S. Cl. 514/424

(57) ABSTRACT

The present invention provides compositions, methods, and kits for treating or preventing a viral infection (e.g., an infection caused by an influenza virus).

### CLAIM OF PRIORITY

This application is a continuation-in-part of U.S. Ser. No. 12/708,076 filed Feb. 18, 2010 and International Patent Application No. PCT/IB2010/000514 filed Feb. 18, 2010 both of which claim priority to U.S. Ser. No. 61/153,418 filed Feb. 18, 2009, the contents of which are fully incorporated herein by reference.

### BACKGROUND OF THE INVENTION

The invention relates to treating viral infections such as influenza.

Diseases caused by viruses are major health problems worldwide, and include many potentially fatal or disabilitating illnesses. Influenza virus, for example, affects 5-15% of the population during epidemics and causes upper respiratory tract infections. Hospitalization and deaths can occur, especially in high-risk groups (elderly, chronically ill and immuno-compromised). Between three and five million cases of severe influenza and between 250,000 and 500,000 deaths are recorded every year around the world. Accordingly, there exists a need for reducing influenza and other viral infections.

Mortality due to influenza is associated with severe lung inflammation. Influenza virus induces several cytokines including interleukin-6, interleukin-8, interleukin-10, tumor necrosis factor-α in the serum and nasopharyngeal fluid. Experiments have demonstrated that mortality associated with influenza infection is due to the ability of the influenza A virus to infect the entire lung and induce high levels of macrophage-derived chemokines and cytokines, which resulted in infiltration of inflammatory cells and severe haemorrhage. It is useful to devise ways of ameliorating influenza with regimens that diminish one or another component of this cytokine response.

Four drugs are currently available for the treatment or prophylaxis of influenza infections: the adamantanes (amantadine and rimantadine) and the newer class of neuraminidase inhibitors (zanamivir [Relenza®] and oseltamivir [Tamiflu®]). The adamantanes interfere with viral uncoating inside the cell. They are effective only against influenza A and are associated with several toxic effects and with rapid emergence of drug-resistant variants. Adamantane-resistant isolates of influenza A are genetically stable, can be transmitted to susceptible contacts, are as pathogenic as wild-type virus isolates, and can be shed for prolonged periods in immunocompromised patients taking the drug. This potential for the development of resistance especially limits the use of the adamantanes for the treatment of influenza, although the drugs still have a place in planning for prophylaxis during an epidemic. However, the Centers For Disease Control (CDC) currently is not recommending amantadine be used because of the poor sensitivity of the virus to this agent.

The neuraminidase inhibitors peramivir, zanamivir, oseltamivir, BCX-1898, BCX-1827, BCX-1989, BCX 1923, BCX 1827 and A315675 interfere with the release of progeny influenza virus from infected host cells, a process that prevents infection of new host cells and thereby halts the spread of infection in the respiratory tract. Since replication of influenza virus in the respiratory tract reaches its peak between 24 and 72 hours after the onset of the illness, drugs such as the neuraminidase inhibitors that act at the stage of viral replication must be administered as early as possible. In contrast to the adamantanes, the neuraminidase inhibitors are associated with very little toxicity and are far less likely to promote the development of drug-resistant influenza. As a class, the neuraminidase inhibitors are effective against all neuraminidase subtypes and, therefore, against all strains of influenza, a key point in epidemic and pandemic preparedness and an important advantage over the adamantanes, which are effective only against sensitive strains of influenza A. These new drugs, if used properly, have great potential for diminishing the effects of influenza infection.

All influenza viruses bear two surface glycoproteins, a hemagglutinin and a neuraminidase, which are the antigens that define the particular strain of influenza. The variation of these molecules over time permits the virus to evade human immune responses and therefore necessitates the formulation of a new vaccine each year. The hemagglutinin is a sialic acid receptor-binding molecule and mediates entry of the virus into the target cell. The neuraminidase—the target molecule of the neuraminidase inhibitor compounds—cleaves the cellular-receptor sialic acid residues to which the newly formed particles are attached. This cleavage releases the viruses, which can now invade new cells. Without neuraminidase, infection would be limited to one round of replication, rarely enough to cause disease. Neuraminidase may also facilitate viral invasion of the upper airways, possibly by cleaving the sialic acid moieties on the mucin that bathes the airway epithelial cells.

The ability of transition-state analogues of sialic acid to inhibit the influenza neuraminidase was first recognized in the 1970s, but the design of highly effective inhibitors became feasible when analysis of the three-dimensional structure of influenza neuraminidase disclosed the location and structure of the catalytic site. Potent inhibitors such as zanamivir closely mimic the natural substrate, fitting into the active site pocket and engaging the protein in the most energetically favorable interaction. Zanamivir is administered by oral inhalation, which delivers the drug directly to the respiratory tract. Oseltamivir was developed through modifications to the sialic acid analogue framework (including the addition of a lipophilic side chain) that allow the drug to be used orally.

Zanamivir is not bioavailable orally and is marketed as a dry powder for inhalation. It is delivered directly to the respiratory tract through an inhaler (Diskhaler, Glaxo Wellcome) that holds small pouches or “blisters” of the drug. Zanamivir is highly concentrated in the respiratory tract; 10 to 20 percent of the active compound reaches the lungs, and the rest is deposited in the oropharynx. Five to 15 percent of the total dose is absorbed and excreted in the urine, resulting in a bioavailability of 2 percent, a feature that is potentially advantageous in situations in which a systemic drug is undesirable. The concentration of the drug in the respiratory tract has been estimated to be more than 1000 times as high as the 50 percent inhibitory concentration (IC₅₀) for neuraminidase; in addition, the inhibitory effect starts within 10 seconds—two favorable features in terms of reducing the likelihood of emergence of drug-resistant variant viruses.

Oseltamivir is available as a capsule or powder for liquid suspension with acceptable oral bioavailability. It is absorbed from the gastrointestinal tract, is converted by hepatic esterases to the active form of the compound (oseltamivir carboxylate), and is widely distributed in the body. The half-life is 6 to 10 hours. The drug is excreted primarily through the kidneys; thus, dosing must be modified in patients with renal insufficiency. Oseltamivir achieves high plasma levels and thus can act outside the respiratory tract.

A key advantage of the neuraminidase inhibitors, and a major difference from the adamantanes, is that development of resistance is less rare. The global neuraminidase inhibitor susceptibility network (NISN), which coordinates the analysis of clinical isolates collected through the World Health Organization's surveillance network, found no influenza isolates with spontaneous resistance to neuraminidase inhibitors. Until recently, there was little emergence of resistance during treatment and no resistant viruses isolated from immunocompetent persons who received zanamivir. For oseltamivir, the published frequency of viruses that were isolated after treatment and were resistant to the drug is somewhat higher. About 0.4 percent of treated adults harbored viruses with resistant neuraminidases.

However, more resistant isolates emerged during treatment of children. One study identified resistant isolates in 4 percent of treated children, and in a recent study of children treated with oseltamivir in Japan, 9 of 50 treated children harbored viruses with mutations in the neuraminidase gene that encoded drug-resistant neuraminidase proteins. If this frequent emergence of resistant mutants is found to be a general occurrence in children, it is a serious concern, especially since children are an important source of the spread of influenza in the community. The most clinically relevant question is whether the oseltamivir-resistant viruses are transmissible and pathogenic. To date, no documented transmission of an oseltamivir-resistant virus has occurred between people. Generally, neuraminidase mutations lead to a functionally defective enzyme, which reduces the fitness of the virus and causes decreased pathogenicity, at least in animal models. However, in the ferret model, resistant variants with the same mutation that is found in some children grew well in both the index ferret and in contact animals and were readily transmitted, raising concern that some oseltamivir-resistant mutant viruses might be transmissible during an epidemic.

Case detection is confounded by the nonspecificity of initial manifestations of illness, so that detailed contact and travel histories and knowledge of viral activity in poultry are essential. Commercial rapid antigen tests are insensitive, and confirmatory diagnosis requires sophisticated laboratory support. Unlike human influenza, avian influenza A (H5N 1) may have higher viral titers in the throat than in the nose, and hence, analysis of throat swabs or lower respiratory samples may offer a more sensitive means of diagnosis. Recent human isolates are fully resistant to M2 inhibitors, and increased doses of oral oseltamivir may be warranted for the treatment of severe illness. Despite recent progress, knowledge of the epidemiology, natural history, and management of influenza A (H5N1) disease in humans is incomplete. There is an urgent need for improving the efficacy if drugs used to treat influenza virus, including the avian variety. The combinations included herein may solve this problem.

U.S. Pat. No. 6,436,971 relates to a method of prophylaxis of or reducing the severity of bacterial infections which occur after a viral infection by administering a PDE4 inhibitor. This invention and its compositions are specifically targeting influenza viral infections and excludes treatment or prophylaxis of bacterial infections associated therewith.

### SUMMARY OF THE INVENTION

We have identified combinations of agents which can reduce mortality rates of mice infected with an influenza virus. On this basis, the present invention provides compositions, methods, and kits useful in treating influenza viral infections.

Accordingly, in a first aspect, the invention features compositions comprising a combination of a neuraminidase inhibitor and a phosphodiesterase inhibitor. The neuraminidase inhibitor may be, for example, oseltamivir, zanamivir, peramivir, or analogs thereof. In one embodiment, the PDE inhibitor is a compound in Table 1 or analogs thereof. In another embodiment, the PDE inhibitor is ibudilast, rolipram, roflumilast or analogs thereof. In yet another embodiment, the composition also includes amantadine or rimantadine. The compounds may be present in an amount sufficient to treat or prevent a viral infection caused by influenza virus (e.g., by any of the influenza types, subtypes, or strains described herein), wherein the influenza virus may or may not be resistant to oseltamivir. In a particular embodiment, the influenza virus may be of type A, B, or C. In another embodiment, the influenza virus may be of subtype H1N1. The composition may be formulated for administration by any route known in the art such as oral, parenteral (e.g., intravenously or intramuscularly), rectal, determatological, cutaneous, nasal, vaginal, inhalant, skin (patch), ocular, intrathecal, and intracranial. In certain embodiments, the composition includes, consists of, or consists essentially of (a) a combination of active ingredients and (b) one or more pharmaceutically acceptable excipients.

In another aspect, the invention features a method for treating or preventing an influenza viral infection in a patient. The method includes administering to the subject an amount of a neuraminidase inhibitor and a PDE inhibitor sufficient to treat or prevent the viral infection in the patient. The neuraminidase inhibitor may be, for example, oseltamivir, zanamivir, peramivir, or analogs thereof. In one embodiment, the PDE inhibitor is a compound in Table 1 or analogs thereof. In another embodiment, the PDE inhibitor is ibudilast, rolipram, roflumilast or analogs thereof. In yet another embodiment, the method includes administering amantadine or rimantadine in combination with a neuraminidase inhibitor and a PDE inhibitor to treat or prevent the viral infection in the patient. In certain embodiments, the neuraminidase inhibitor, PDE inhibitor, and (if present) amantadine or rimantine are administered within 7 days, 1 day, or 1 hour of each other or substantially simultaneously.

The invention also features kits. One kit includes (a) a neuraminidase inhibitor; (b) a PDE inhibitor; and (c) instructions for administering (a) and (b) to a patient for treating or preventing an influenza viral infection.

Another kit includes (a) a neuraminidase inhibitor; and (b) instructions for administering (a) with at least one PDE inhibitor to a patient for treating or preventing an influenza viral infection.

Yet another kit includes (a) a PDE4 inhibitor; and (b) instructions for administering (a) with at least one neuraminidase inhibitor to a patient for treating or preventing an influenza viral infection.

Another kit includes (a) a neuraminidase inhibitor; (b) a PDE inhibitor; (c) amantadine or rimantadine; and (d) instructions for administering (a), (b), and (c) to a patient for treating or preventing an influenza viral infection.

Another kit includes (a) a neuraminidase inhibitor; (b) a PDE inhibitor; and (c) instructions for administering (a) and (b) with amantadine or rimantadine to a patient for treating or preventing an influenza viral infection.

By a “neuraminidase inhibitor” is meant any compound that can substantially inhibit the activity of one or more neuraminidases in vitro or in vivo or any member of the class of compounds having an IC₅₀ of 100 μM or lower concentration for a neuraminidase. Exemplary neuraminidase inhibitors for use in the invention include oseltamivir, zanamivir, perarnivir, and analogs thereof, and are described herein.

By a “neuraminidase” is meant an enzyme that can cleave the glycosidic linkage of neuraminic acid, which has the following structure:

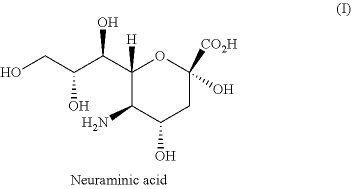

By a “PDE inhibitor” is meant any compound that can substantially inhibit the activity of one or more PDEs in vitro or in vivo or any member of the class of compounds having an IC₅₀ of 100 μM or lower concentration for a PDE. When a PDE inhibitor is described herein as having activity against a particular type of PDE, the inhibitor may also have activity against other types, unless otherwise stated. Exemplary PDE inhibitors for use in the invention are described herein.

By a “PDE” is meant an enzyme of the phosphodiesterase superfamily, including but not limited to any member of the 11 phosphodiesterase families (e.g. 1, 2, 3, 4, 5, 6, 7, 8, 9, 10, and 11), or any enzyme that can degrade the 3′ phosphodiester bond in cyclic adenosine monophosphate (cAMP) or cyclic guanodine monophosphate (cGMP).

By “substantially inhibit” is meant to abrogate the catalytic activity of an enzyme or reduce said catalytic activity by at least 1%, 5%, 10%, 20%, 30%, 50%, 70%, 80%, 90%, 95%, or 99%, as determined by a suitable assay, as compared to activity in the absence of the target.

By “substantially simultaneously” is meant that compounds are administered at a time(s) such that two or more administered compounds can interact together in a manner which enhances antiviral activity.

To “treat” is meant to administer one or more agents to measurably slow or stop the replication of a virus in vitro or in vivo, to measurably decrease the load of a virus (e.g., any virus described herein including an influenza virus) in a cell in vitro or in vivo, or to reduce at least one symptom (e.g., inflammation) associated with having a viral infection in a patient. Desirably, the slowing in replication, the decrease in viral load, or reduction in the symptom is at least 20%, 30%, 50%, 70%, 80%, 90%, 95%, or 99%, as determined using a suitable assay (e.g., a inflammation assay described herein) as compared to in the absence of the agent.

To “prevent” a disease is meant to reduce to frequency of appearance of the disease in a population of patients, the likelihood of an individual patient developing the disease, or to reduce the symptoms or severity of a disease upon its appearance by administering one or more agents to a patient prior to diagnosis of the disease or manifestation of disease symptoms.

By “an effective amount” is meant the amount of an agent, alone or in combination with another therapeutic regimen, required to treat a patient with a viral infection (e.g., caused by any virus described herein including an influenza virus) in a clinically relevant manner. A sufficient amount of an agent used to practice the present invention for therapeutic treatment of conditions caused by a virus varies depending upon the manner of administration, the age, body weight, and general health of the patient. Ultimately, the prescribers will decide the appropriate amount and dosage regimen. Additionally, an effective amount may be an amount of an agent in a combination of the invention that is safe and efficacious in the treatment of a patient having a viral infection over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular agent formulated for a given route of administration for treatment of any human disease or condition. For example, a low dosage of an agent that treats a viral infection and that is formulated for administration by intravenous injection will differ from a low dosage of the same agent formulated for oral administration.

By a “high dosage” is meant at least 5% (e.g., at least 10%, 20%, 50%, 100%, 200%, 300%, 500%, 1,000%, 2,000%, 5,000%, or 10,000%) more than the highest standard recommended dosage of a particular agent for treatment of any human disease or condition.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the agents of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “anti-influenza” or “antiviral” include those compounds which prevent the release of the virus from leaving a human or other species cell type. These include, but are not limited to peramivir (BCX-1812, RWJ-270201), amantadine, rimantadine, osteltamivir and zanamivir.

The term “M2 inhibitors” refers to compounds or agents which inhibit viral replication and proliferation by blocking an M2 channel present in influenza A viruses. M2 inhibitors include, but are not limited to, amantadine, rimantadine, and pharmaceutically acceptable salts thereof.

The term “mean”, when preceding a pharmacokinetic value (e.g., mean Peak) represents the arithmetic mean value of the pharmacokinetic value unless otherwise specified.

As used herein and in the appended claims, the singular forms “a” “an” and “the” also includes plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

The term “about” generally means within 10%, preferably within 5%, and more preferably within 1% of a given value or range.

Conditions or disorders caused or related to influenza include any condition or disorder in a subject that is caused by, complicated by, or aggravated by the virus. Such conditions or disorders include, but are not limited to, those caused by viruses of the influenza family, including but not limited to, human influenza virus, avain influenza virus, or both.

As used herein, the term “treat” includes one or more of the following:
(a) arresting, delaying the onset (i.e., the period prior to clinical manifestation of a disorder) and/or reducing the risk of developing or worsening a disorder;
(b) relieving or alleviating at least one symptom of a disorder in a mammal, including for example, hypercalcemia; or
(c) relieving or alleviating the intensity and/or duration of a manifestation of a disorder experienced by a mammal including, but not limited to, those which are in response to a given stimulus (e.g., pressure, tissue injury or cold temperature). The term “treat” also includes prophylactically preventing, curing, healing, alleviating, relieving, altering, remedying, ameliorating, improving, or affecting a condition (e.g., a disease), the symptoms of the condition, or the predisposition toward the condition.

The term “Minimum Inhibitory Concentration” means the plasma level of active agent required to inhibit replication of the virus, in vivo or in vitro. It means that quantity sufficient to treat, prevent, cure or mitigate infection with influenza viruses in their hosts or in vitro.

The term “neuraminidase inhibitor” refers to actives which inhibit the viral enzyme neuraminidase, and includes, but is not limited to peramivir, osteltamivir, zanamivir, and A315675.

The terms “anti-viral agents,” “anti-influenza viral agents,” refer to active agents used to inhibit replication or prevent infection with both human and avian influenza viruses, including, but not limited to rimatadine, amantadine, peramivir, zanamivir, oseltamivir, A315675, and their pharmaceutically acceptable salts or prodrugs.

The terms “influenza,” or “influenza virus,” “virus,” or “viral” refer to human, avian and “swine” or H1N1 influenza virus of all strains or genotypes.“Genotypes” includes any biologically active sequence of DNA that is found in an influenza virus.

The term “influenza” refers to an acute viral infection of the respiratory tract caused by a strain of the influenza virus (e.g. influenza virus A, B and C).

The Term “Serum Concentration” or “Serum Concentration Curve” is the graphic representation of the amount of drug in an animal's (including humans) plasma at particular points in time.

The term “Bioavailability” or “F” means the percentage of drug reaching the systemic circulation. Generally, 100% bioavailability occurs with intravenous infusions since drug is delivered directly into the animal. Because of metabolism, first pass effects, food effect, and the like, oral bioavailability is generally lower. The delivery agents described herein have the ability to increase oral bioavailability above the norm of that particular active agent without the delivery agent

The term “Area Under the Curve” or “Area Under the Concentration Curve” means that space present beneath the line of the graphical representation of plasma concentrations versus time in subject(s).

The term “Synergistic Anti-influenza Effect” means a result that is more favorable when anti-influenza agents are administered in combination than when administered alone.

Compounds useful in the invention include those described herein in any of their pharmaceutically acceptable forms, including isomers such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 12 ring carbon atoms, inclusive. Exemplary cyclic groups include cyclopropyl, cyclobutyl, cyclopentyl, and cyclohexyl groups. An alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups.

Other features and advantages of the invention will be apparent from the following Detailed Description, the drawings, and the claims.

### BRIEF DESCRIPTION OF THE DRAWINGS

FIG. 1 is a graph showing survival data for C57/BL6 mice administered with either the combination of oseltamivir and a PDE4 inhibitor or oseltamivir alone in the lethal infection of Influenza A/NWS/33 (H1N1).

FIG. 2 is a graph showing mean day to death for C57/BL6 mice administered with the either the combination of oseltamivir and a PDE4 inhibitor or oseltamivir alone in the lethal infection of Influenza A/NWS/33 (H1N1).

### DETAILED DESCRIPTION OF THE INVENTION

The invention features methods, compositions, and kits for the administration of an effective amount of a combination including a neuraminidase inhibitor and a PDE inhibitor to treat a viral infection.

In particular, we have shown that the neuraminidase inhibitor oseltamivir, in combination with any one of the PDE inhibitors ibudilast, rolipram, and roflumilast, can be used to reduce the mortality associated with an influenza viral infection in mice. On this basis, the invention features methods for treating or preventing influenza viral infections, using a neuraminidase inhibitor in combination with a PDE inhibitor. The invention also features compositions including a neuraminidase inhibitor and a PDE inhibitor, and kits including a neuraminidase inhibitor and a PDE inhibitor. The invention is described in greater detail below.

### Influenza Types, Subtypes, and Strains

The invention relates to the treatment of an influenza viral disease. Influenza viruses are RNA viruses of the family Orthomyxoviridae. Three types of influenza viruses (types A, B, and C) have been identified. Subtypes of type A are based on variations in the hemagglutinin (HA) polypeptide and the neuraminidase (N) polypeptide. Fifteen (H1, H2, H3, H4, H5, H6, H7, H8, H9, H10, H11, H12, H13, H14, and H15) different HA subtypes have been identified, and nine (N1, N2, N3, N4, N5, N6, N7, N8, and N9) N subtypes have been identified. Strains including these subtypes can occur in various combinations (e.g., H1N1, H2N2, H3N2, H5N1, H7N7, H1N2, H9N2, H7N2, H7N3, H10N7). One serotype of influenza B has been identified, and influenza type C is generally less virulent that types A or B.

### Influenza Symptoms

Influenza is characterized by fever, headache, tiredness, cough, sore throat, runny or stuffy nose, body aches, and diarrhea and vomiting. Complications which can develop from an influenza infection include bacterial pneumonia, dehydration, and worsening of chronic medical conditions, such as congestive heart failure, asthma or diabetes. Sinus problems and ear infections can also develop.

Mortality due to influenza infection is often associated with lung inflammation, which can be severe. Influenza virus can induce cytokines including interleukin-6, interleukin-8, interleukin-10, and tumor necrosis factor-alpha in the serum and nasopharyngeal fluid (Laurent et. al., J Med Virol 64:262-268, 2001; Hayden et. al., J Clin Investig 101:643-649, 1998). Mortality associated with influenza infection is often due to the ability of the influenza A virus to infect the entire lung and induce high levels of macrophage-derived chemokines and cytokines, which results in infiltration of inflammatory cells and severe haemorrhage (Kobasa et. al., Nature 431:703-707, 2004).

### Compounds

Certain compounds that may be employed as agents in the methods, compositions, and kits of the present invention are discussed in greater detail below. It will be understood that analogs or pharmaceutically acceptable salts of any these compound can be used in the methods, compositions, and kits of the present invention.

### Neuraminidase Inhibitors

The compositions, methods, and kits of the invention can include a neuraminidase inhibitor or an analog thereof. Neuraminidase inhibitors are a class of compounds which block viral neuraminidase peptide, preventing viral replication from the host cell. Neuraminidase inhibitors act against both influenza type A and type B. Suitable neuraminidase inhibitors include oseltamivir, zanamivir, and peramivir.

Non-limiting examples of Neuraminidase Inhibitors (NIs) include, but are not limited to, peramivir (also referred to as BCX-1812 or RWJ-270201, manufactured by BioCryst Pharmaceuticals), osteltamivir (available as Tamiflu® from Roche), zanamivir® (available as Relenza® from GlaxoSmithKline), A315675 (being researched by Abbot Laboratories), BCX-1827, BCX-1989, BCX 1923, and BCX 1827.

NIs can be combined with PDE4Is to form a pharmaceutical composition of the present invention. For example, pharmaceutical compositions of the present invention include peramivir or osteltamivir or zanamivir or A315675 and a PDE4I disclosed herein. In one embodiment, a pharmaceutical comprises osteltamivir and D159687.

Oseltamivir

In certain embodiments, oseltamivir ((3R,4R,5S)-4-acetylamino-5-amino-3(1-ethylpropoxy)-1-cyclohexene-1-carboxylic acid, ethyl ester; e.g. oseltamivir phosphate) or its structural analogs may be used in the compositions, methods, and kits of the invention. Oseltamivir has the following structure:

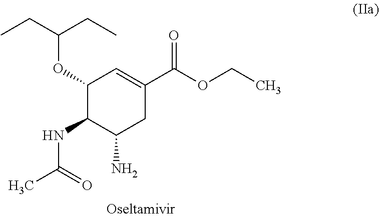

Oseltamivir is a prodrug, which is hydrolyzed hepatically to the active metabolite, the free carboxylate of oseltamivir (GS4071), which has the following structure:

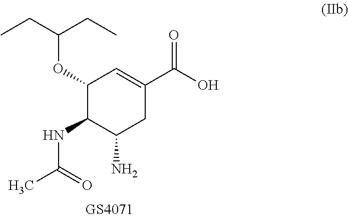

Oseltamivir and GS4071 are described in U.S. Pat. No. 5,763,483.

Oseltamivir can be administered as an oral tablet. The standard recommended dosage of oseltamivir for the treatment or prevention of influenza is 75 mg twice daily for 5 days. Dosages for children and patients with renal impairment are decreased and vary by body weight.

Structural analogs of oseltamivir include those having the formula:

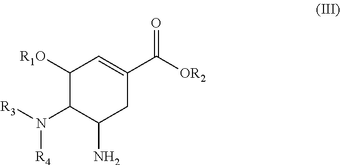

wherein R₁ is an alkyl group or a substituted alkyl group, R₂ is an alkyl group, and R₃ and R₄ are, independently, H or a substituent of an amino group, wherein R₃ and R₄ are not both H. Additional information regarding these oseltamivir analogs can be found in U.S. Pat. No. 6,437,171.

Additional structural analogs of oseltamivir include those having the formula:

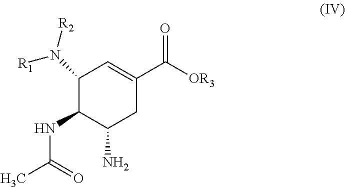

wherein R₁ and R₂ are described below:

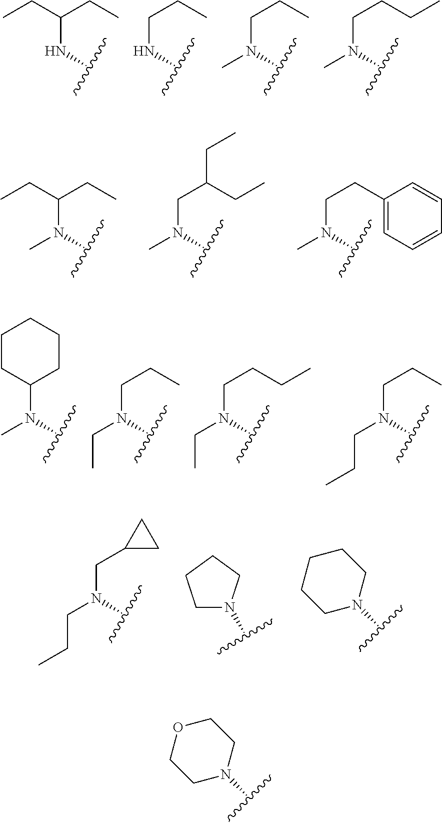

and R₃ is H or CH₂CH₃. Additional information regarding these oseltamivir analogs can be found in U.S. Pat. No. 6,111,132. Additional oseltamivir analogs, synthetic intermediates, and methods of synthesis can be found in U.S. Pat. Nos. 6,057,459, 6,204,398, 6,225,341, 6,376,674, 6,455,571, 6,518,305, 6,518,438,6,593,314, and 7,122,684, each of which is incorporated by reference.

Zanamivir

In certain embodiments, zanamivir ((2R,3R,4S)-4-[(diaminomethylidene)amino]-3-acetamido-2-[(1R,2R)-1,2,3-trihydroxypropyl]-3,4-dihydro-2H-pyran-6-carboxylic acid) or its structural analogs may be used in the compositions, methods, and kits of the invention. Zanamivir has the following structure:

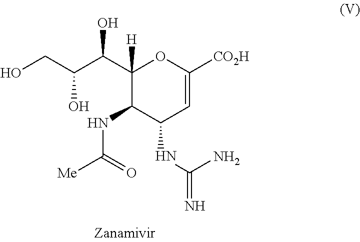

Zanamivir can be administered through oral inhalation using a breath-activated plastic device called a Diskhaler. The standard recommended dosage of zanamivir for the treatment of influenza is 10 mg (2 inhalations) twice daily for 5 days in patients 7 years and older. Zanamivir can also be used to prevent influenza infection for patients 5 years and older with a standard recommended dosage of 1 inhalation per day for 10 to 28 days. Zanamivir is not recommended for people with underlying respiratory disease such as asthma or chronic obstructive pulmonary disease. Zanamivir has not been shown to shorten the duration of influenza in people with these diseases, and some people have had serious side effects of bronchospasm (wheezing) and worsening lung function.

Structural analogs of zanamivir includes compounds having the formula:

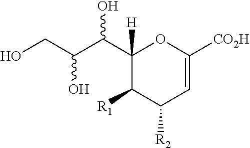

wherein [symbol] indicates lack of a specified stereochemistry; and R₁ is (alk)ₓNR₃R₄, CN or N₃; where alk is an unsubstituted or substituted methylene; x is 0 or 1; R₃ is H, C₁₋₆ alkyl, aryl, aralkyl, amidine, NR₄R₅ or an unsaturated or saturated ring containing one or more heteroatoms selected from the group consisting of nitrogen, oxygen and sulfur; R₄ is H, C₁₋₆ alkyl, or allyl; R₅ is H or C₁₋₆ alkyl; and R₂ is NNHCOR₆ where R₆ is H, substituted or unsubstituted C₁₋₄ alkyl or aryl or a pharmaceutically acceptable salt thereof. The compound may have the following stereochemistry:

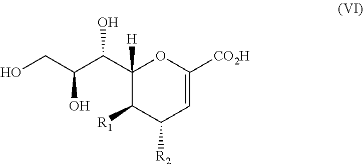

Zanamivir is made by Glaxo Smith Kline under the name Relenza® and is currently administered by inhalation only. This product is approved to treat type A and B influenza, the two types most responsible for flu epidemics. Clinical studies showed that for the drug to be effective, patients needed to start treatment within two days of the onset of symptoms. The drug seemed to be less effective in patients whose symptoms weren't severe or didn't include a fever. Relenza is a powder that is inhaled twice a day for five days from a breath-activated plastic device called a Diskhaler.

Additional information regarding these zanamivir analogs can be found in U.S. Pat. No. 5,360,817. Additional zanamivir analogs, synthetic intermediates, and methods of synthesis are described in U.S. Pat. Nos. 5,859,284, 5,866,601, 5,886,213, 5,958,973, 5,985,859, 5,944,377, 6,114,386, 6,225,341, 6,340,702, and 6,451,766, each of which is incorporated by reference.

Peramivir

In certain embodiments, peramivir ((1S,2S,3S,4R)-3-[(1S)-1-Acetamido-2-ethyl-butyl]-4-(diaminomethylideneamino)-2-hydroxy-cyclopentane-1-carboxylic acid), its structural analogs, or pharmaceutically acceptable salts thereof, may be used in the compositions, methods, and kits of the invention. Peramivir has the following structure:

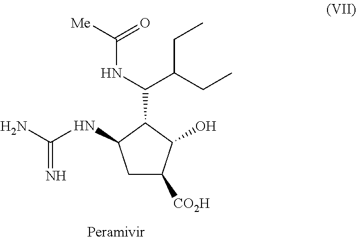

Structural analogs of peramivir includes compounds having the formula:

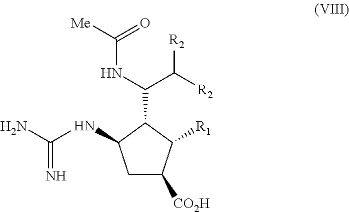

wherein R₁ is H or OH and R₂ are both CH₂CH₃ or both CH₂CH₂CH₃.

Additional information regarding these peramivir analogs can be found in WO2007/095218 and additional peramivir analogs are described in WO2007/087056 and which are hereby incorporated by reference in its entirety. Peramivir is also described in U.S. Pat. No. 5,453,533, which is hereby incorporated by reference in its entirety. Peramivir interferes with the expulsion of viral particles from cells by inhibiting neurinamidase enzyme. The cyclopentane peramivir (BCX-1812, RWJ-270201) is a highly selective inhibitor of influenza A and B virus neuraminidases and a potent inhibitor of influenza A and B virus replication in cell culture. The in vitro potency appears to be greater than either zanamivir or oseltamivir carboxylate based on the generally lower EC(50) values.

Peramivir in combination with other antiretroviral agents may be used for the treatment of human influenza virus, and perhaps including avian influenza virus. In Phase I studies, peramivir was well-tolerated, with single or multiple oral doses up to 800 mg/kg/day evaluated. In clinical trials with patients experimentally infected with influenza A or B viruses, oral treatment with peramivir significantly reduced nasal wash virus titers with no adverse effects. Phase III clinical trials are underway as of February, 2006.

The recommended dose of the injectable form of peramivir has not yet established, but may be around 800 mg/kg/day daily or higher as an injection, such as an intramuscular injection. As known to applicants, positive results from orally administered peramivir have not been obtained. Embodiments of the present invention, however, provide an oral pharmaceutical composition comprising peramavir and at least one of the delivery agents disclosed herein.

### PDE Inhibitors

A PDE inhibitor is a compound which can inhibit the enzymatic activity of one or more of the subtypes of the enzyme phosphodiesterase (PDE), therefore preventing the inactivation of the intracellular second messengers cAMP or cGMP.

PDE inhibitors may be employed in combination with a neuraminidase to treat an influenza viral infection. Exemplary PDE inhibitors for use in the invention are shown in Table 1.

TABLE 1
PDE Inhibitors

| Compound | Synonym | PDE Activity |
|---|---|---|
| 256066 | | 4 |
| 349U85 | 6-piperidino-2(1H)-quinolinone | 3 |
| 5E3623 | | |
| A 021311 | | |
| A 906119 | | |
| Adibendan | 5,7-dihydro-7,7-dimethyl-2-(4-pyridinyl)- | 3 |
| | pyrrolo(2,3-f)benzimidazol-6(1H)-one, | |
| AWD-12-281 | | 4 |
| Amlexanox | 2-amino-7-isopropyl-5-oxo-5H- | 3, 4 |
| | [1]benzopyrano[2,3-b]pyridine-3-carboxylic acid | |
| | (U.S. Pat. No. 4,143,042) | |
| Amrinone | 5-Amino-(3,4′-bipyridin)-6(1H)-one | 3, 4 |
| Anagrelide | 6,7-dichloro-1,5-dihydroimidazo | 3, 4 |
| | (2,1-b)quinazolin-2(3H)-one | |
| AN-2728 | | 4 |
| AP-0679 | | 4 |
| AP 155 | 2-(1-piperazinyl)-4H-pyrido[1,2-a]pyrimidin-4- | 4 |
| | one | |
| AR 12456 | CAS Reg. No. 100557-06-0 | 4 |
| Arofylline | 3-(4-chlorophenyl)-3,7-dihydro-1-propyl-1H- | 4 |
| | Purine-2,6-dione | |
| ASP-9831 | | 4 |
| Ataquimast | 1-ethyl-3-(methylamino)-2(1H)-quinoxalinone | 3 |
| Atizoram | tetrahydro-5-[4-methoxy-3-[(1S,2S,4R)-2- | 4 |
| | norbornyloxy]phenyl]- | |
| | 2(1H)-pyrimidinone | |
| Atopik | | 4 |
| (Barrier Therapeutics) | | |
| ATZ 1993 | 3-carboxy-4,5-dihydro-1-[1-(3- | |
| | ethoxyphenyl)propyl]-7-(5-pyrimidinyl)methoxy- | |
| | [1H]-benz[g]indazole (Teikoku Hormone) | |
| Avanafil | 4-{[(3-chloro-4-methoxyphenyl)methyl]amino}- | 5 |
| | 2-[(2S)-2- | |
| | (hydroxymethyl)pyrrolidin-1-yl]-N-(pyrimidin-2- | |
| | ylmethyl)pyrimidine- | |
| | 5-carboxamide | |
| AVE 8112 | | 4 |
| AWD 12171 | | 5 |
| AWD 12187 | | 7 |
| AWD 12250 | | 5 |
| AWD12343 | | 4 |
| BAY 38-3045 | | 1 |
| BAY 60-7550 (Alexis | 2-(3,4-Dimethoxybenzyl)-7-[(1R)-1-[(1R)-1- | 2 |
| Biochemicals) | hydroxyethyl]-4-phenylbutyl]-5- | |
| | methylimidazo[5,1-f][1,2,4]triazin-4(3H)-one | |
| BAY 61-9987 (Bayer | | 4 |
| AG) | | |
| BBB 022 | | 4 |
| BCX-1993 | | |
| BCX-1812 (RWJ- | | |
| 270201): | | |
| Bemarinone | 5,6-dimethoxy-4-methyl-2(1H)-quinazolinone | 3 |
| Bemoradan | 6-(3,4-dihydo-3-oxo-1,4(2H)-benzoxazin-7-yl)- | 3 |
| | 2,3,4,5-tetrahydro-5-methylpyridazin-3-one | |
| Benafentrine | (6-(p-acetamidophenyl)-1,2,3,4,4a,10b- | 3, 4 |
| | hexahydro-8,9-dimethoxy-2-methyl- | |
| | benzo[c][1,6]naphthyridine | |
| BFGP 385 | | |
| BMY 20844 | 1,3-dihydro-7,8-dimethyl-2H-imidazo[4,5- | 4 |
| | b]quinolin-2-one | |
| BMY 21190 | | 4 |
| BMY 43351 | 1-(cyclohexylmethyl)-4-(4-((2,3-dihydro-2-oxo- | 4 |
| | 1H-imidazo(4,5-b)quinolin-7-yl)oxy)-1- | |
| | oxobutyl)-Piperazine | |
| BRL 50481 | 3-(N,N-dimethylsulfonamido)-4-methyl- | 7 |
| | nitrobenzene | |
| BY 244 | | |
| C 3885 | | 4 |
| Caffeine citrate | 2-hydroxypropane-1,2,3-tricarboxylic acid | 4 |
| CBS-3595 | | 4 |
| CC 10004 | N-(2-((1S)-1-(3-ethoxy-4-methoxyphenyl)-2- | 4 |
| (Apremilast) | (methylsulfonyl)ethyl)-2,3-dihydro-1,3-dioxo- | |
| | 1H-isoindol-4-yl)-Acetamide | |
| CC 1088 | | 4 |
| CC 11050 | | 4 |
| CC 3052 | | 4 |
| CC 7085 | | 4 |
| CCT 62 | 6-[(3-methylene-2-oxo-5-phenyl-5- | 3 |
| | tetrahydrofuranyl) methoxy]quinolinone | |
| CDC 998 | | 4 |
| CDP 840 | 4-((2R)-2-(3-(cyclopentyloxy)-4- | 4 |
| | methoxyphenyl)-2-phenylethyl)-Pyridine | |
| CGH 2466 | 2-amino-4-(3,4-dichlorophenyl)-5-pyridin-4-yl- | 4 |
| | thiazol | |
| CH 2874 | | 4 |
| CH 3442 | | 4 |
| CH 3697 | | 4 |
| CH 4139 | | 4 |
| CH 422 | | 4 |
| CH 673 | | 4 |
| CH 928 | | 4 |
| CI 1018 | N-(3,4,6,7-tetrahydro-9-methyl-4-oxo-1- | 4 |
| | phenylpyrrolo(3,2,1-jk)(1,4)benzodiazepin-3-yl)- | |
| | 4-Pyridinecarboxamide | |
| CI 1044 | N-[9-amino-4-oxo-1-phenyl-3,4,6,7- | 4 |
| | tetrahydropyrrolo[3,2,1-jk][1,4]benzodiazepin- | |
| | 3(R)-yl]pyridine-3-carboxamide | |
| CI 930 | 4,5-dihydro-6-[4-(1H-imidazol-1-yl)phenyl]-5- | 3 |
| | methyl-3(2H)-pyridazinone | |
| Cilomilast (Ariflo ®) | U.S. Pat. No. 5,552,438, 4-cyano-4-(3- | 4 |
| | cyclopentyloxy-4-methoxy-phenyl)cyclohexane- | |
| | 1-carboxylic acid | |
| Cilostamide | N-cyclohexyl-4-((1,2-dihydro-2-oxo-6- | 3 |
| | quinolinyl)oxy)-N-methyl-butanamide | |
| Cilostazol | 6-[4-(1-cyclohexyl-1H-tetrazol-5-yl)butoxy]-3,4- | 3, 4 |
| | dihydro-2(1H)-quinolinone; U.S. Pat. No. | |
| | 4,277,479 | |
| Cipamfylline | 8-amino-1,3-bis(cyclopropylmethyl)-3,7-dihydro- | 4 |
| | 1H-Purine-2,6-dione | |
| CK 3197 | 2H-imidazol-2-one, 1-benzoyl-5-(4-(4,5-dihydro- | |
| | 2-methyl-1H-imidazol-1-yl)benzoyl)-4-ethyl-1,3- | |
| | dihydro | |
| CP 146523 | 4′-Methoxy-3-methyl-3′-(5-phenyl-pentyloxy)- | 4 |
| | biphenyl-4-carboxylic acid | |
| CP 166907 | | |
| CP 220629 | 1-cyclopentyl-3-ethyl-6-(2-methylphenyl)-7-oxo- | 4 |
| | 4,5,6,7-tetrahydro-1H-pyrazolo[3,4-c]pyridine | |
| CP 293121 | (S)-3-(3-Cyclopentyloxy-4-methoxy)phenyl-2- | 4 |
| | isoxazoline-5-hydroxamic acid | |
| CP 293321 | | 4 |
| CP 248 | (Z)-5-fluoro-2-methyl-1-[p- | 2 |
| | (methylsulfonyl)benzylidene]indene-3-acetic acid | |
| CP 353164 | 5-(3-Cyclopentyloxy-4-methoxy-phenyl)- | 4 |
| | pyridine-2-carboxylic acid amide | |
| CP 77059 | | |
| CRC-3590 | | 4 |
| CT 1579 | | |
| CT 1786 | | |
| CT 2820 | | |
| CT-5357 | | 4 |
| D 157140 | 1-[[4-methoxy-3-(3-nitrophenyl)phenyl]methyl]- | 4 |
| | 1,2,4-triazole | |
| D 158681 | 2-fluoro-1-[(4-fluorophenyl)methyl]-4-methoxy- | 4 |
| | 3-(3-nitrophenyl)benzene | |
| D 159382 | [4-[[2-fluoro-4-methoxy-3-(3- | 4 |
| | nitrophenyl)phenyl]methyl]phenyl]methylurea | |
| D159404 | [4-(3′-chloro-6-methoxy-biphenyl-3-ylmethyl)- | 4 |
| | phenyl)-urea | |
| D 159687 | [3-(3′-Chloro-6-methoxy-biphenyl-3-ylmethyl)- | 4 |
| | phenyl]-urea | |
| D 159153 | [4-[[2-fluoro-4-methoxy-3-(3- | 4 |
| | nitrophenyl)phenyl]methyl]phenyl]urea | |
| D 22888 | 8-methoxy-5-N-propyl-3-methyl-1-ethyl- | 4 |
| | imidazo [1,5-a]-pyrido [3,2-e]-pyrazinone | |
| D 4418 | N-(2,5-dichloro-3-pyridinyl)-8-methoxy-5- | 4 |
| | Quinolinecarboxamide | |
| Dasantafil | 7-(3-bromo-4-methoxyphenylmethyl)-1-ethyl-8- | 5 |
| | {[(1R,2R)-2-hydroxycyclopentyl] = amino}-3- | |
| | (2-hydroxyethyl)-3,7-dihydro-1H-purine-2,6- | |
| | dione | |
| Daxalipram | | 4 |
| Dipyridamole | 2-{[9-(bis(2-hydroxyethyl)amino)-2,7-bis(1- | 5, 6, 7, 8, 10, |
| | piperidyl)-3,5,8,10-tetrazabicyclo[4.4.0]deca- | 11 |
| | 2,4,7,9,11-pentaen-4-yl]-(2- | |
| | hydroxyethyl)amino}ethanol | |
| DE-103 | | 4 |
| DG-071 | | |
| DN 9693 | 1,5-Dihydro-7-(1-piperidinyl)-imidazo[2,1- | 4 |
| | b]quinazolin-2(3H)-one dihydrochloride hydrate | |
| Doxofylline | 7-(1,3-dioxolan-2-ylmethyl)-1,3-dimethyl-3,7- | 4 |
| | dihydro-1H-purine-2,6-dione (U.S. Pat. No. | |
| | 4,187,308) | |
| DWP-205 derivatives | | 4 |
| E 4010 | 4-(3-chloro-4-metoxybenzyl)amino-1-(4- | 5 |
| | hydroxypiperidino)-6-phthalazinecarbonitrile | |
| | monohydrochloride | |
| E 4021 | sodium 1-[6-chloro-4-(3,4- | 4, 5 |
| | methylenedioxybenzyl)aminoquinazolin-2- | |
| | yl]piperidine-4-carboxylate sesquihydrate | |
| EHNA | erythro-9-(2-hydroxy-3-nonyl)adenine | 2, 3, 4 |
| EHT 0202 | 3,7-dimethyl-1-(5-oxohexyl)purine-2,6-dione | 4 |
| ELB 353 | | 4 |
| EMD 53998 | 5-(1-(3,4-dimethoxybenzoyl)- | 3 |
| | 1,2,3,4-tetrahydro-6-quinolyl)-6-methyl-3,6- | |
| | dihydro- | |
| | 2H-1,3,4-thiadiazin-2-one | |
| EMD 57033 | (+)-5-[1-(3,4-dimethoxybenzoyl)-3,4-dihydro- | 3 |
| | 2H-quinolin-6-yl]-6-methyl-3,6-dihydro-1,3,4- | |
| | thiadiazin-2-one | |
| EMD 57439 | (−)-5-[1-(3,4-dimethoxybenzoyl)-3,4-dihydro-2H- | 3 |
| | quinolin-6-yl]-6-methyl-3,6-dihydro-1,3,4- | |
| | thiadiazin-2-one | |
| EMD 82639 | | 5 |
| EMR 62203 | | 5 |
| Enoximone | 4-methyl-5-{[4- | 3 |
| | (methylsulfanyl)phenyl]carbonyl}-2,3-dihydro- | |
| | 1H-imidazol-2-one | |
| Enprofylline | 3-propyl xanthine | 4 |
| ER 017996 | 4-((3,4-(methylenedioxy)benzyl)amino)-6,7,8- | |
| | trimethoxyquinazoline | |
| Etazolate | 1-ethyl-4-((1-methylethylidene)hydrazino)-1h- | 4 |
| | pyrazolo(3,4-b) pyridine-5-carboxylic acid | |
| Exisulind | (1Z)-5-Fluoro-2-methyl-1-[[4- | 2, 5 |
| | (methylsulfonyl)phenyl]methylene]-1H-indene- | |
| | 3-acetic acid | |
| Filaminast | (1E)-1-(3-(cyclopentyloxy)-4-methoxyphenyl)- | 4, 7 |
| | Ethanone O-(aminocarbonyl)oxime | |
| FR 226807 | N-(3,4-dimethoxybenzyl)-2-{[(1R)-2-hydroxy-1- | 5 |
| | methylethyl]amino}-5-nitrobenzamide | |
| FR 229934 | | 5 |
| GI 104313 | 6-{4-[N-[-2-[3-(2-cyanophenoxy)-2- | 3 |
| | hydroxypropylamino]-2- | |
| | methylpropyl]carbamoylmethoxy-3- | |
| | chlorophenyl]}-4,5-dihydro-3(2H) pyridazinone | |
| GRC 3015 | | 4 |
| GRC 3566 | | 4 |
| GRC 3785 | | 4 |
| GRC 4039 | | 4 |
| GSK 256066 | | 4 |
| GW 3600 | (7aS,7R)-7-(3-cyclopentyloxy-4- | 4 |
| | methoxyphenyl)-7a-methyl-2,5,6,7,7a-penta- | |
| | hydro-2-azapyrrolizin-3-one | |
| GW 842470 | N-(3,5-dichloro-4-pyridinyl)-1-((4- | 4 |
| | fluorophenyl)methyl)-5-hydroxy-.alpha.-oxo-1H- | |
| | Indole-3-acetamide | |
| Helenalin | (3aS,4S,4aR,7aR,8R,9aR)-4-hydroxy-4a,8- | 5 |
| | dimethyl-3-methylidene-3,3a,4,4a,7a,8,9,9a- | |
| | octahydroazuleno[6,5-b]furan-2,5-dione | |
| HFV 1017 | | |
| HT-0712 | | 4 |
| Hydroxypumafentrine | | 4 |
| IBMX | 3-isobutyl-1-methylxanthine | 3, 4, 5 |
| Ibudilast | 1-(2-isopropyl-pyrazolo[1,5-a]pyridine-3-yl)-2- | Not selective |
| | methylpropan-1-one (U.S. Pat. No. 3,850,941) | |
| IC 485 | | 4 |
| Iosartan derivatives | | 4 |
| IPL 423088 | | |
| IPL 455903 | (3S,5S)-5-(3-cyclopentyloxy-4-methoxy- | 4 |
| | phenyl)-3-(3-methyl-benzyl)-piperidin-2-one | |
| Isbufylline | 1,3-dimethyl-7-isobutylxanthine | 4 |
| IWF 12214 | | |
| K 123 | | |
| KF 17625 | 5-phenyl-1H-imidazo(4,5-c)(1,8)naphthyridin- | 4 |
| | 4(5H)-one | |
| KF 19514 | 5-phenyl-3-(3-pyridil) methyl-3H-imidazo[4,5- | 1, 4 |
| | c][1,8]naphthyridin-4(5H)-one | |
| KF 31327 | 3-ethyl-8-[2-[4-(hydroxymethyl)piperidin-1- | 5 |
| | yl]benzylamino]-2,3--dihydro-1H-imidazo[4,5- | |
| | g]quinazoline-2-thione | |
| KF 31334 | | |
| Ks-505a | 1-carboxy- | 1 |
| | 2,3,4,4a,4b,5,6,6a,6b,7,8,8a,8b,9,10,10a, | |
| | 14,16,17,17a,17b,18,19,19a,19b, | |
| | 20,21,21a,21b,22,23,23a-dotriacontahydro-14- | |
| | hydroxy-8a,10a-bis(hydroxymethyl)-14-(3- | |
| | methoxy-3-oxopropyl)-1,4,4a,6,6a,17b,19b,21b- | |
| | octamethyl beta-D-glucopyranosiduronic acid | |
| KT 734 | | 5 |
| KW 4490 | | 4 |
| L 686398 | 9-[1,S,2R)-2- | 3, 4 |
| | fluoro-1-methylpropyl]-2-methoxy-6-(1- | |
| | piperazinyl]- | |
| | purine hydrochloride | |
| L 787258 (Merck Frosst | | 4 |
| Canada) | | |
| L 826141 | 4-{2-(3,4-bis-difluromethoxyphenyl)-2-{4- | 4 |
| | (1,1,1,3,3,3-hexafluoro-2-hydroxypropan-2-yl)- | |
| | phenyl]-ethyl}-3-methylpyridine-1-oxide | |
| L 869298 | (+)-1\|(S)-(+)-3-{2-[(3-Cyclopropyloxy-4- | 4 |
| | difluromethoxy)-phenyl]-2-[5-(2-(1-hydroxy-1- | |
| | trifluoromethyl-2,2,2-trifluoro)ethyl)- | |
| | thiazolyl]ethyl}pyridine N-Oxide | |
| L-869299 | (−)-1\|(R)-(−)-3-{2-[(3-cyclopropyloxy-4- | 4 |
| | difluromethoxy)phenyl]-2-[5-(2-(1-hydroxy-1- | |
| | trifluoromethyl-2,2,2- | |
| | trifluoro)ethyl)thiazolyl]ethyl}pyridine N-Oxide | |
| Laprafylline | 8-[2-[4-(dicyclohexylmethyl)piperazin-1- | 4 |
| | yl]ethyl]-1-methyl-3-(2-methylpropyl)-7H- | |
| | purine-2,6-dione | |
| LAS 34179 | | 5 |
| LAS 37779 | | 4 |
| Levosimendan | (−)-0-cyano-N-{4-[(4R)-4-methyl-6-oxo-1,4,5,6- | 3 |
| | tetrahydropyridazin-3- | |
| | yl]phenyl}methanecarbohydrazonoyl cyanide | |
| Lirimilast | Methanesulfonic acid 2-(2,4- | 4 |
| | dichlorophenylcarbonyl)-3-ureidobenzo-furan-6- | |
| | yl ester | |
| Lixazinone | N-cyclohexyl-N-methyl-4-((1,2,3,5-tetrahydro-2- | 3, 4 |
| | oxoimidazo(2,1-b)quinazolin-7-yl)oxy)- | |
| | Butanamide | |
| LPDE4 inhibitor (Bayer) | | 4 |
| Macquarimicin A | | |
| MEM 1414 | | 4 |
| MEM 1917 | | 4 |
| MERCK1 | (5R)-6-(4-{[2-(3-iodobenzyl)-3-oxocyclohex-1- | 3 |
| | en-1-yl]amino}phenyl)-5-methyl-4,5- | |
| | dihydropyridazin-3(2H)-one\| | |
| | dihydropyridazinone | |
| Mesopram | (5R)-5-(4-methoxy-3-propoxyphenyl)-5-methyl- | 4 |
| | 2-Oxazolidinone | |
| Milrinone | 6-dihydro-2-methyl-6-oxo-3,4′-bipyridine)-5- | 3, 4 |
| | carbonitrile; U.S. Pat.t No. 4,478,836 | |
| MIMX | 1 8-methoxymethyl-3-isobutyl-1-methylxantine | 1 |
| MK 0952 | | 4 |
| MKS 213492 | | |
| MN 001 | 4-[6-acetyl-3-[3-(4-acetyl-3-hydroxy-2- | 4 |
| | propylphenylthi-o)propoxy]-2- | |
| | propylphenoxy]butyric acid | |
| Mopidamol | U.S. Pat. No. 3,322,755 | 4 |
| MS 857 | 4-acetyl-1-methyl-7-(4-pyridyl)-5,6,7,8- | 3 |
| | tetrahydro-3(2H)-isoquinolinone | |
| MX 2120 | | |
| N 3601 | | |
| Nanterinone | 6-(2,4-dimethyl-1H-imidazol-1-yl)-8-methyl- | 3 |
| | 2(1H)-quinolinone | |
| NCS 613 | | 4 |
| ND 1251 | | 4 |
| ND 1510 | | 4 |
| ND7001 | | 2 |
| Nestifylline | 7-(1,3-dithiolan-2-ylmethyl)-1,3-dimethylpurine- | |
| | 2,6-dione | |
| NIK 616 | | 4 |
| NIP 520 | | 3 |
| NM 702 | | 5 |
| NSP 306 | | 3 |
| NSP 513 | | 3 |
| NSP 804 | 4,5-dihydro-6-[4-[(2-methyl-3-oxo-1- | 3 |
| | cyclopentenyl)-amino] phenyl]-3(2H)- | |
| | pyridazinone | |
| NSP 805 | 4,5-dihydro-5-methyl-6-[4-[(2-methyl-3-oxo-1- | 3 |
| | cyclopentenyl) amino]phenyl]-3(2H)- | |
| | pyridazinone | |
| NVP ABE 171 | | 4 |
| Nycomed's PDE4 | | 4 |
| inhibitor | | |
| Oglemilast | N-(3,5-Dichloropyridin-4-yl)-4-difluoromethoxy- | 4 |
| | 8-((methylsulfonyl)amino)dibenzo(b,d)furan-1- | |
| | carboxamide | |
| Olprinone | 5-imidazo[2,1-f]pyridin-6-yl-6-methyl-2-oxo- | 3, 4 |
| | 1H-pyridine-3-carbonitrile | |
| ONO 1505 | 4-[2-(2-hydroxyethoxy)ethylamino]-2-(1H- | 5 |
| | imidazol-1-yl)-6-methoxy-quinazoline | |
| | methanesulphonate | |
| ONO 6126 | | 4 |
| OPC 33509 | (−)-6-[3-[3-cyclopropyl-3-[(1R,2R)-2- | 3 |
| | hydroxyclohexyl]ureido]-propoxy]-2(1H)- | |
| | quinolinone | |
| OPC 33540 | 6-[3-[3-cyclooctyl-3-[(1R[*],2R[*])-2- | 3 |
| | hydroxycyclohexyl]ureido]-propoxy]-2(1H)- | |
| | quinolinone | |
| ORG 20241 | N-hydroxy-4-(3,4-dimethoxyphenyl)-thiazole-2- | 3, 4 |
| | carboximidamide | |
| ORG 20494 | | |
| ORG 30029 | N-hydroxy-5,6-dimethoxy-benzo[b]thiophene-2- | 3, 4 |
| | carboximide hydrochloride | |
| ORG 9731 | 4-fluoro-N-hydroxy-5,6-dimethoxy- | 3, 4 |
| | benzo[b]thiophene-2-carboximidamide | |
| | methanesulphonate | |
| ORG 9935 | 4,5-dihydro-6-(5,6-dimethoxy-benzo[b]-thien-2- | 3 |
| | yl)-methyl-1-(2H)-pyridazinone | |
| OSI 461 | N-benzyl-2-[(3Z)-6-fluoro-2-methyl-3-(pyridin- | 5 |
| | 4-ylmethylidene)inden-1-yl]acetamide | |
| | hydrochloride | |
| Osthole | 7-methoxy-8-(3-methyl-2-butenyl)-2H-1- | 5 |
| | benzopyran-2-one | |
| Ouazinone | (R)-6-chloro-1,5-dihydro-3-methyl-imidazo[2,1- | 3 |
| | b]quinazolin-2-one | |
| PAB 13 | 6-bromo-8-(methylamino)imidazo[1,2-a]pyrazine | |
| PAB 15 | 6-bromo-8-(ethylamino)imidazo[1,2-a]pyrazine | |
| PAB 23 | 3-bromo-8-(methylamino)imidazo[1,2-a]pyrazine | |
| Papaverine | 1-[(3.4-dimethoxyphenyl)-methyl]-6,7- | 5, 6, 7, 10 |
| | dimethoxyisoquinolone | |
| PDB 093 | | 4 |
| Pentoxifylline | 3,7-dimethyl-1-(5-oxohexyl)-3,7-dihydropurine- | |
| | 2,6-dione | |
| Piclamilast | 3-cyclopentyloxy-N-(3,5-dichloropyridin-4-yl)-4- | 4, 7 |
| | methoxy-benzamide | |
| Pimobendan | 6-[2-(4-methoxyphenyl)-1H-benzimidazol-5-yl]- | 3, 4 |
| | 5-methyl-4,5-dihydropyridazin-3(2H)-one | |
| Piroximone | 4-ethyl-1,3-dihydro-5-(4-pyridinylcarbonyl)-2H- | 3 |
| | imidazol-2-one | |
| Plexxikon's PDE4 | | 4 |
| inhibitor | | |
| Prinoxodan | 6-(3,4-dihydro-3-methyl-2-oxoquinazolinyl)-4,5- | |
| | dihydro-3-pyridazinone | |
| Propentofylline | 3-methyl-1-(5-oxohexyl)-7-propyl-3,7-dihydro- | 5 |
| | 1H-purine-2,6-dione | |
| Pumafentrine | rel-(M)-4-((4aR,10bS)-9-ethoxy-1,2,3,4,4a,10b- | 4 |
| | hexahydro-8-methoxy-2-methylbenzo(c)(1, | |
| | 6)naphthyridin-6-yl)-N,N-bis(1-methylethyl)- | |
| | Benzamide | |
| R 1627 | | 4 |
| R 79595 | N-cyclohexyl-N-methyl-2-[[[phenyl (1,2,3,5- | 3 |
| | tetrahydro-2 oxoimidazo [2,1-b]-quinazolin-7-yl) | |
| | methylene] amin] oxy] acetamide | |
| REN 1053 | | |
| Revizinone | (E)-N-cyclohexyl-N-methyl-2-(((phenyl(1,2,3,5- | 3 |
| | tetrahydro-2-oxoimidazo(2,1-b)quinazolin-7- | |
| | yl)methylene)amino)oxy)-Acetamide | |
| Ro20-1724 | 4-(3-butoxy-4-methoxybenzyl)-2- | 4 |
| | imidazolidinone | |
| Roflumilast | 3-(cyclopropylmethoxy)-N-(3,5-dichloro-4- | 4, 5 |
| | pyridinyl)-4-(difluoromethoxy)-Benzamide | |
| Rolipram | 4-(3-cyclopentyloxy-4-methoxyphenyl)-2- | 4 |
| | pyrrolidone; U.S. Pat. No. 4,193,926 | |
| RP 116474 | | |
| RPL554 | 9,10-dimethoxy-2(2,4,6-trimethylphenylimino)- | 3, 4 |
| | 3-(N-carbamoyl-2-aminoethyl)-3,4,6,7- | |
| | tetrahydro-2H-pyrimido[6,1-a]isoquinolin-4-one | |
| RPL565 | 6,7-dihydro-2-(2,6-diisopropylphenoxy)-9,10- | 3, 4 |
| | dimethoxy-4H-pyrimido[6,1-a]isoquinolin-4-one | |
| RPR 114597 | | 4 |
| RPR 117658 | | 4 |
| RPR 122818 | | 4 |
| RPR 132294 | | 4 |
| RPR 132703 | | 4 |
| RS-25344 | | 4 |
| Saterinone | 1,2-dihydro-5-(4-(2-hydroxy-3-(4-(2- | 3 |
| | methoxyphenyl)-1-piperazinyl)propoxy)phenyl)- | |
| | 6-methyl-2-oxo-3-pyridinecarbonitrile | |
| Satigrel | 4-cyano-5,5-bis(4-methoxyphenyl)-4-pentenoic | 2, 3, 5 |
| | acid; U.S. Pat. No. 4,978,767 | |
| SCA 40 | 6-Bromo-8-methylaminoimidazol[1,2- | 3 |
| | a]pyrazine-2carbonitrile | |
| SCH 351591 | N-(3,5-Dichloro-1-oxido-4-pyridinyl)-8- | 4 |
| | methoxy-2-(trifluoromethyl)-5-quinoline | |
| | Carboxamide | |
| SCH 45752 | | |
| SCH 46642 | | 5 |
| SCH 51866 | cis-5,6a,7,8,9,9a-hexahydro-2-(4- | 1, 5 |
| | (trifluoromethyl)phenylmethyl)-5-methyl- | |
| | cyclopent (4,5)imidazo(2,1-b)purin-4(3H)-one | |
| SCH 51866 | cis-5,6a,7,8,9,9a-hexahydro-2-[4- | 1, 5 |
| | (trifluoromethyl)phenylmethyl]-5-methyl- | |
| | cyclopent[4,5]imidazo[2,1-b]purin-4(3H)-one | |
| SCH 59498 | cis-2-hexyl-5-methyl-3,4,5,6a,7,8,9,9a- | 5 |
| | octahydrocyclopent[4,5]imidazo-[2,-1-b]purin- | |
| | 4-one | |
| SDZ ISQ 747 | | 4 |
| SDZ ISQ 844 | 6,7-dimethoxy-1-(3,4-dimethoxyphenyl)-3- | 3, 4 |
| | hydroxymethyl-3,4-dihydroisoquinoline | |
| SDZ MKS 492 | R(+)-(8-[(1-(3,4-Dimethoxyphenyl)-2- | 3 |
| | hydroxyethyl)amino]-3,7-dihydro-7-(2- | |
| | methoxyethyl)-1,3-dimethyl-1H-purine-2,6-dione | |
| Senazodan | | 3 |
| Siguazodan | N-Cyano-N′-methyl-N″-[4-(1,4,5,6-tetrahydro- | 3, 4 |
| | 4-Methyl-6-oxo-3-pyridazinyl)phenyl]guanidine | |
| Sildenafil | U.S. Pat. No. 5,250,534, 5-[2-ethoxy-5-(4- | 5 |
| | methyl-1-piperazinylsulfonyl)phenyl]-1-methyl- | |
| | 3-n-propyl-1,6-dihydro-7H-pyrazolo[4,3- | |
| | d]pyrimidin-7-one | |
| SK 3530 | | 5 |
| SKF 94120 | 5-(4-acetamidophenyl)pyrazin-2(1H)-one | 3 |
| SKF 95654 | ±-5-methyl-6-[4-(4-oxo-1,4-dihydropyridin-1- | 3 |
| | yl)phenyl]-4,5-dihydro-3(2H)-pyridazinone | |
| SKF 96231 | 2-(2-propoxyphenyl)-6-purinone | 3, 4, 5 |
| SKF 107806 | | 4 |
| SLX 2101 | | 5 |
| Sulmazole | 1H-Imidazo(4,5-b)pyridine, 2-(2-methoxy-4- | 3 |
| | (methylsulfinyl)phenyl)-2-((2-methoxy-4- | |
| | methylsulfinyl)phenyl)-H-imidazo(4,5-b)pyridine | |
| T 0156 | 2-(2-Methylpyridin-4-yl)methyl-4-(3,4,5- | 5 |
| | trimethoxyphenyl)-8-(pyrimidin-2-yl)methoxy- | |
| | 1,2-dihydro-1-oxo-2,7-naphthyridine-3- | |
| | carboxylic acid methyl ester hydrochloride | |
| T 1032 | methyl2-(4-aminophenyl)-1,2-dihydro-1-oxo-7- | 5 |
| | (2-pyridylmethoxy)-4-(3,4,5-trimethoxyphenyl)- | |
| | 3-isoquinoline carboxylate sulfate | |
| T 440 | 6,7-Diethoxy-1-[1-(2-methoxyethyl)-2-oxo-1,2- | 4 |
| | dihydropyridin-4-yl]naphthalene-2,3-dimethanol | |
| Tadalafil | (6R,12aR)-6-(1,3-benzodioxol-5-yl)-2-methyl- | 4, 5 |
| | 2,3,6,7,12,12a- | |
| | hexahydropyrazino[1,2,1,6]pyrido[3,4-b]indole- | |
| | 1,4-dione | |
| Tetomilast | 6-(2-(3,4-diethoxyphenyl)-4-thiazolyl)-2- | 4 |
| | Pyridinecarboxylic acid | |
| Theophylline | 3,7-dihydro-1,3-dimethyl-1H-purine-2,6-dione | Not selective |
| Tibenelast | 5,6-diethoxybenzo(B)thiophene-2-carboxylic | 4 |
| | acid | |
| Tipelukast | | 4 |
| Toborinone | (+/−)-6-[3-(3,4-dimethoxybenzylamino)-2- | 3 |
| | hydroxypropoxy]-2(1H)-quinolinone | |
| Tofimilast | 9-cyclopentyl-7-ethyl-6,9-dihydro-3-(2-thienyl)- | 4 |
| | 5H-Pyrazolo(3,4-c)-1,2,4-triazolo(4,3-a)pyridine | |
| Tolafentrine | N-[4-[(4aS,10bR)-8,9-dimethoxy-2-methyl- | 3, 4 |
| | 3,4,4a,10b-tetrahydro-1H-pyrido[4,3- | |
| | c]isoquinolin-6-yl]phenyl]-4- | |
| | methylbenzenesulfonamide | |
| Torbafylline | 7-(ethoxymethyl)-3,7-dihydro-1-(5-hydroxy-5- | 4 |
| | methylhexyl)-3-methyl-1-H-purine-2,6-dione | |
| Trequinsin | 2,3,6,7-tetrahydro-9,10-dimethoxy-3-methyl-2- | 2, 3, 4 |
| | ((2,4, 6-trimethylphenyl)imino)-4H-pyrimido(6, | |
| | 1-a)isoquinolin-4-one | |
| Trombodipine | | |
| UCB 29936 | | 4 |
| UDCG 212 | 5-methyl-6-[2-(4-oxo-1-cyclohexa-2,5- | 3 |
| | dienylidene)-1,3-dihydrobenzimidazol-5-yl]-4,5- | |
| | dihydro-2H-pyridazin-3-one | |
| Udenafil | 3-(1-methyl-7-oxo-3-propyl-4H-pyrazolo[5,4- | 5 |
| | e]pyrimidin-5-yl)-N-[2-(1-methylpyrrolidin-2- | |
| | yl)ethyl]-4-propoxybenzenesulfonamide | |
| UK 114542 | 5-[2-ethoxy-5-(morpholinylacetyl) phenyl]-1,6- | 5 |
| | dihydro-1-methyl-3-propyl-7H-pyrazolo [4,3-d]- | |
| | pyrimidin-7-one | |
| UK 343664 | 3-ethyl-5-(5-((4-ethylpiperazino)sulphonyl)-2- | 5 |
| | propoxyphenyl)-2-(2-pyridylmethyl)-6,7- | |
| | dihydro-2H-pyrazolo(4,3-d)pyrimidin-7-one | |
| UK 357903 | 1-ethyl-4-{3-[3-ethyl-6,7-dihydro-7-oxo-2-(2- | 5 |
| | pyridylmethyl)-2H-pyrazolo[4,3-d] pyrimidin-5- | |
| | yl]-2-(2-methoxyethoxy)5-pyridylsulphonyl} | |
| | piperazine | |
| UK 369003 | | 5 |
| UK 66838 | | |
| V 11294A | 3-((3-(cyclopentyloxy)-4- | 4 |
| | methoxyphenyl)methyl)-N-ethyl-8-(1- | |
| | methylethyl)-3H-Purin-6-amine | |
| | monohydrochloride | |
| Vardenafil | 2-(2-Ethoxy-5-(4-ethylpiperazin-1-yl-1- | 5 |
| | sulfonyl)phenyl)-5-methyl-7-propyl-3H- | |
| | imidazo(5,1-f)(1,2,4)triazin-4-one | |
| Vasotrope | | |
| Vesnarinone | 6-[4-(3,4-dimethoxybenzoyl)piperazin-1-yl]-3,4- | 3, 5 |
| | dihydro-1H-quinolin-2-one | |
| Vinpocetine | (3-alpha,16-alpha)-eburnamenine-14-carboxylic | 1, 3, 4 |
| | acid ethyl ester | |
| WAY 122331 | 1-aza-10-(3-cyclopentyloxy-4-methoxyphenyl)- | 4 |
| | 7,8-dimethyl-3-oxaspiro[4.5]dec-7-en-2-one | |
| WAY 126120 | | 4 |
| WAY 127093B | [(3S)-3-(3-cyclopentyloxy-4methoxyphenyl)-2- | 4 |
| | methyl-5-oxopyrazolidinyl]-N-(3- | |
| | pyridylmethyl)carboxamide | |
| WIN 58237 | 1-cyclopentyl-3-methyl-6-(4-pyridinyl)pyrazolo | 5 |
| | (3,4-d)pyrimidin-4(5H)-one | |
| WIN 58993 | 5-methyl-6-pyridin-4-yl-3H-[1,3]thiazolo[5,4- | 3 |
| | e]pyridin-2-one | |
| WIN 62005 | 5-methyl-6-pyridin-4-yl-1,3-dihydroimidazo[4,5- | 3 |
| | e]pyridin-2-one | |
| WIN 62582 | 6-pyridin-4-yl-5-(trifluoromethyl)-1,3- | 3 |
| | dihydroimidazo[4,5-b]pyridin-2-one | |
| WIN 63291 | 6-methyl-2-oxo-5-quinolin-6-yl-1H-pyridine-3- | 3 |
| | carbonitrile | |
| WIN 65579 | 1-cyclopentyl-6-(3-ethoxy-4-pyridinyl)-3-ethyl- | 5 |
| | 1,7-dihydro-4H-pyrazolo[3,-4-d]pyrimidin-4- | |
| | one | |
| XT-044 | | 4 |
| XT-611 | | 4 |
| Y 20487 | 6-(3,6-dihydro-2-oxo-2H-1,3,4-thiadiazin-5-yl)- | 3 |
| | 3,4-dihydro-2(1H)-quinolinone | |
| YM 58997 | 4-(3-bromophenyl)-1,7-diethylpyrido[2,3- | 4 |
| | d]pyrimidin-2(1H)-one | |
| YM 976 | 4-(3-chlorophenyl)-1,7-diethylpyrido(2,3- | 4 |
| | d)pyrimidin-2(1H)-one | |
| Z 15370A | | 4 |
| Zaprinast | 1,4-Dihydro-5-(2-propoxyphenyl)-7H-1,2,3- | 5 |
| | triazolo[4,5-d]pyrimidine-7-one | |
| Zaprinast | 2-o-propoxyphenyl-8-azapurine-6-one | 1, 5 |
| Zardaverine | 6-(4-(difluoromethoxy)-3-methoxyphenyl)- | 3, 4 |
| | 3(2H)-Pyridazinone | |
| Zindotrine | 8-methyl-6-(1-piperidinyl)-1,2,4-triazolo(4,3- | |
| | b)pyridazine | |

Other PDE 1 inhibitors are described in U.S. Patent Application Nos. 20040259792 and 20050075795, incorporated herein by reference. Other PDE 2 inhibitors are described in U.S. Patent Application No. 20030176316, incorporated herein by reference. Other PDE 3 inhibitors are described in the following patents and patent applications: EP 0 653 426, EP 0 294 647, EP 0 357 788, EP 0 220 044, EP 0 326 307, EP 0 207 500, EP 0 406 958, EP 0 150937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference. Other PDE 4 inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; International Publication No. WO 92/079778; and Molnar-Kimber, K. L. et al. J. Immunol., 150:295A (1993), each of which is incorporated herein by reference. Other PDE 5 inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference. Other PDE 6 inhibitors that can be used in the methods, compositions, and kits of the invention include those described in U.S. Patent Application Nos. 20040259792, 20040248957, 20040242673, and 20040259880, each of which is incorporated herein by reference. Other PDE 7 inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; International Publication WO 00/68230; and Martinez et al., J. Med. Chem. 43:683-689 (2000), each of which is incorporated herein by reference. Other PDE inhibitors that can be used in the methods, compositions, and kits of the invention are described in U.S. Pat. No. 6,953,774.

Additional PDE Inhibitors, especially PDE4 inhibitors, polynucleotides, the polypeptides and isoforms they encode, antibodies and specific binding partners thereto, which may be useful in this invention are include in U.S. Pat. No. 7,696,198, U.S. Pat. No. 7,655,802, U.S. Pat. No. 7,585,882, U.S. Pat. No. 7,576,080, U.S. Pat. No. 7,495,017, U.S. Pat. No. 7,432,266, U.S. Pat. No. 7,405,230, U.S. Pat. No. 7,342,021, U.S. Pat. No. 7,335,654, U.S. Pat. No. 7,332,486, U.S. Pat. No. 7,235,579, U.S. Pat. No. 7,226,930, U.S. Pat. No. 7,205,320, U.S. Pat. No. 7,153,871, U.S. Pat. No. 7,087,625, U.S. Pat. No. 6,699,890, U.S. Pat. No. 6,656,717, and U.S. Patent Application Nos. 2009/0324569, 2009/0136473, 2009/0131530, 2009/0130077, 2009/0130076, US20100029689, US20100010017, US20090054434, US20070093510, US20060168668, US20060166316, US20050289660, US20050289660, US20040102460, US20040087584, US20030149052, US20030139406, US20030045533, US20020151566, and International Publication Nos. WO 2010/003084, WO 2009/067607, WO 2009/029214, WO 2007/123953, WO 2007/103554, WO 2007/103370, WO 2007/103260, WO 2007/100880, WO2007098214, WO2007098169, WO2007022280, WO06135828, WO06071988, WO06044955, WO06044528, WO06028957, WO05061458, WO04094411, WO04094375, WO04090126, WO 04/089285, WO 04/094375, WO04046113, WO04014913, WO9828740, WO04014911, WO04009557, WO04009552, WO03044170, WO03032981, WO02098878, WO02074726, WO0100851, and foreign application and patent nos. EP1549619, EP1529049, EP1435944 and DE102007022945 the entire disclosures of which are incorporated herein by reference.

Further PDE4 inhibitors useful in this invention are disclosed by Burgun, et. al. (Nature Biotechnology Vol. 28 (1):63-72. January 2010), with special emphasis on compound D159687.

Ibudilast

In certain embodiments, ibudilast or an ibudilast analog, as defined by formula below, may be used in the compositions, methods, and kits of the invention.

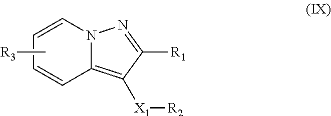

In this formula, R₁ and R₂ are each, independently, selected from H, C₁₋₇ alkyl, C₂₋₇ alkenyl, C₂₋₇alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₃ is selected from H, halide, alkoxy, and C₁₋₄ alkyl; X₁ is selected from C═O, C═N—NH—R₄, C═C(R₅)—C(O)—R₆, C═CH═CH—C(O)—R₆, and C(OH)—R₇; R₄ is selected from H and acyl; R₅ is selected from H, halide, and C₁₋₄ alkyl; R₆ is selected from OH, alkoxy and amido; and R₇ is selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. Compounds of formula (VI) include, the compounds described in U.S. Pat. Nos. 3,850,941; 4,097,483; 4,578,392; 4,925,849; 4,994,453; and 5,296,490. Commercially available compounds of formula (VI) include ibudilast and KC-764.

The standard recommended dosage for the treatment of bronchial asthma is typically 10 mg of ibudilast twice daily, while in the case of cerebrovascular disorders, the standard recommended dosage is 10 mg of ibudilast three times daily. The structure of ibudilast is shown below:

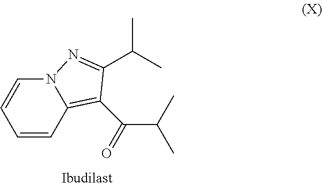

KC-764 (CAS 94457-09-7) is reported to be a platelet aggregation inhibitor. The structure of KC-764 is shown below:

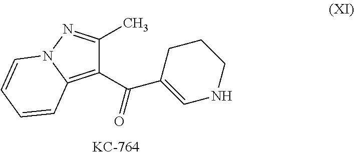

KC-764 and other compounds of formula (VI) can be prepared using the synthetic methods described in U.S. Pat. Nos. 3,850,941; 4,097,483; 4,578,392; 4,925,849; 4,994,453; and 5,296,490.

Roflumilast

In one embodiment of the invention, an antiviral agent is administered or formulated with roflumilast (3-(cyclopropylmethoxy)-N-(3,5-dichloropyridin-4-yl)-4-(difluoromethoxy)benzamide). Roflumilast has the following structure:

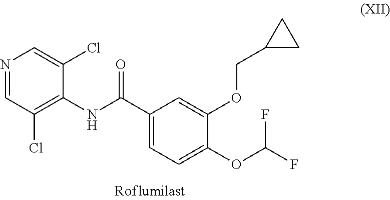

Rolipram

In one embodiment of the invention, an antiviral agent is administered or formulated with rolipram (4-[3-(cyclopentyloxy)-4-methoxyphenyl]-2-pyrrolidone) or an analog of rolipram. Rolipram has the following structure:

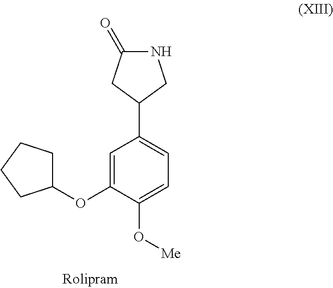

Rolipram analogs are described by formula (I) of U.S. Pat. No. 4,193,926, hereby incorporated by reference.

Other PDE Inhibitors Structures

The structures of additional PDE inhibitors are given in Table 2:

TABLE 2
Structures of additional PDE inhibitors

| | |
|---|---|
| AWD-12-281 | 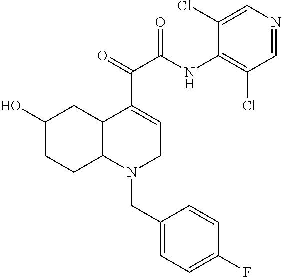 |
| AN-2728 | 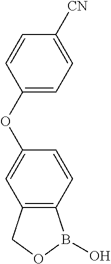 |
| arofylline | 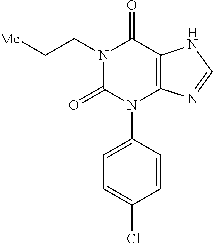 |
| CDP-840 | 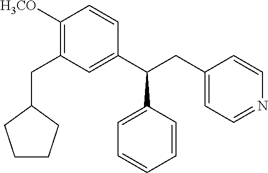 |
| Cilomilast | 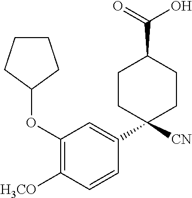 |
| Cipamfylline | 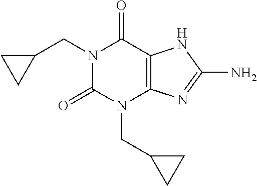 |
| D22888 | 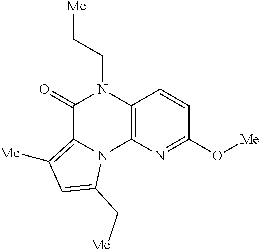 |
| Daxalipram | 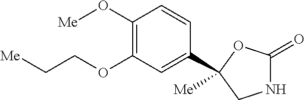 |
| Doxophylline | 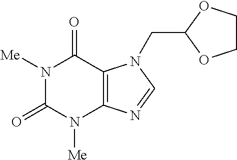 |
| HT-0712 | 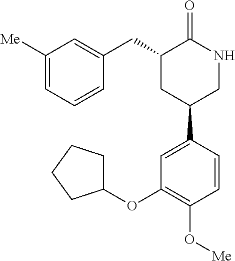 |
| Org-30029 | 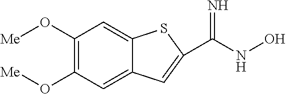 |
| Piclamilast | 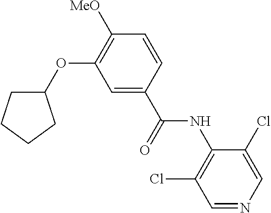 |
| Pumafentrine | 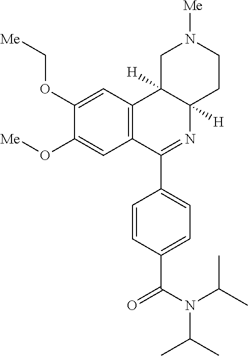 |
| Ro-20-1724 | 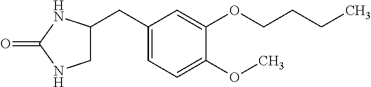 |
| RPL 554 | 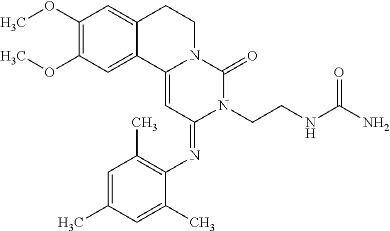 |
| SCH-351591 | 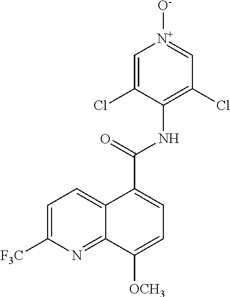 |
| SDZ ISQ 844 | 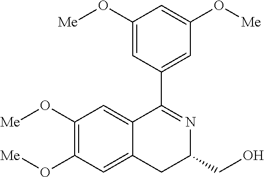 |
| Tetomilast | 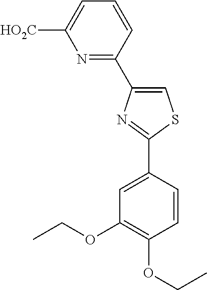 |
| Tipelukast | 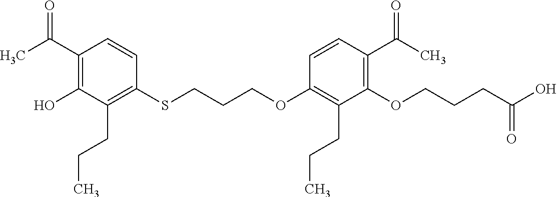 |
| Tolafentrine | 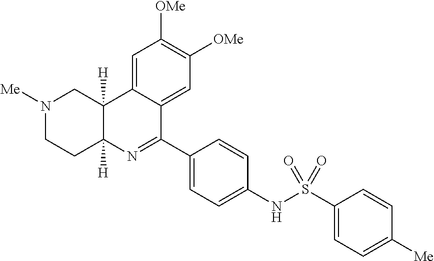 |
| Theophylline | 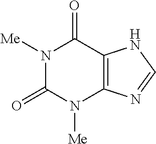 |

### Amantadine and Rimantadine

The compositions, methods, and kits of the invention can include amantadine (e.g. amantadine hydrochloride), rimantadine (e.g. rimantadine hydrochloride), or analogs thereof. The structures of amantadine and rimantadine are given below:

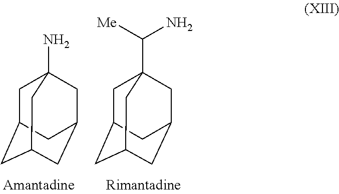

Amantadine (adamantan-1-amine) and rimantadine (1-(adamantan-1-yl)ethan- 1-amine) are substituted adamantane compounds which can be used singly for the treatment or prevention of influenza A. Currently the mechanism of viral inhibition by amantadine and rimantadine is not well understood. These compounds are believed to inhibit influenza's viral replication by binding to the viral M2 ion channel.

The recommended dose for amantadine or rimantadine is 100 mg taken twice daily. If the patient does not respond to this dosage, then the dosage may be increased to 200 mg, or to a maximum of 300 mg. A reduction in dosage to 100 mg/day of rimantadine is recommended for persons who have severe hepatic dysfunction or those with creatinine clearance less than 10 mL/min. Other persons with less severe hepatic or renal dysfunction taking 100 mg/day or rimantadine should be observed closely, and the dosage should be reduced or the drug discontinued, if necessary.

In certain embodiments, amantadine, rimantadine, and analogs thereof can be used in combination with a neuraminidase inhibitor and a PDE inhibitor in the compositions, methods and kits of the invention. Amantadine analogs include compounds having the formula (XIV):

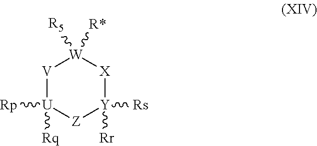

wherein R* is -(A)ₙ-(CR₁R₂)ₘ—NR₃R₄, n+m=0, 1, or 2, A is selected from the group consisting of linear or branched C ₁-C₆ alkyl, linear or branched C₂-C₆ alkenyl, and linear or branched C₂-C₆ alkynyl, R₁ and R₂ are independently selected from the group consisting of hydrogen, linear or branched C₁-C₆ alkyl, linear or branched C₂-C₆ alkenyl, linear or branched C₂-C₆ alkynyl, aryl, substituted aryl, and arylalkyl, R₃ and R₄ are independently selected from the group consisting of hydrogen, linear or branched C₁-C₆ alkyl, linear or branched C₂-C₆ alkenyl, and linear or branched C₂-C₆ alkynyl, or together form C₂-C₁₀ alkylene or C₂-C₆ alkenylene or together with the N form a 3-7-membered azacycloalkane or azacycloalkene, including substituted (C₁-C₆ alkyl, C₂-C₆ alkenyl) 3-7-membered azacycloalkane or azacycloalkene; or independently R₃ or R₄ may join with Rₚ, R_{q}, Rᵣ, or Rₛ to form an alkylene chain —CH(R₆)—(CH₂)ₜ—, wherein t=0 or 1 and the left side of the alkylene chain is attached to U or Y, the right side of the alkylene chain is attached to N, and R₆ is selected from the group consisting of hydrogen, linear or branched C₁-C₆ alkyl, linear or branched C₂-C₆ alkenyl, linear or branched C₂-C₆ alkynyl, aryl, substituted aryl and arylalkyl; or independently R₃ or R₄ may join with R₅ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N; R₅ is selected from the group consisting of hydrogen, linear or branched C₁-C₆ alkyl (C₁-C₆), linear or branched C₂-C₆ alkenyl, and linear or branched C₂-C₆ alkynyl, or R₅ combines with the carbon to which it is attached and the next adjacent ring carbon to form a double bond, Rₚ, R_{q}, Rᵣ, and Rₛ are independently selected from the group consisting of hydrogen, linear or branched C₁-C₆ alkyl, linear or branched C₂-C₆ alkenyl, linear or branched C₂-C₆ alkynyl, C₃-C₆ cycloalkyl, aryl, substituted aryl, and arylaklyl, or Rₚ, R_{q}, Rᵣ, or Rₛ independently may form a double bond with U or with Y or to which it is attached, or Rₚ, R_{q}, Rᵣ, or Rₛ may combine together to represent a lower alkylene —(CH₂)ₓ— or a lower alkenylene bridge wherein x is 2-5, inclusive, which alkylene bridge may, in turn, combine with R₅ to form an additional lower alkylene —(CH₂)_{y}— or a lower alkenylene bridge, wherein y is 1 -3, inclusive, U, V, W, X, Y, Z represent carbon atoms, and include optical isomers, diastereomers, polymorphs, enantiomers, hydrates, pharmaceutically acceptable salts, and mixtures of compounds within formula (I).

The ring defined by U-V-W-X-Y-Z is preferably selected from the group consisting of cyclohexane, cyclohex-2-ene, cyclohex-3-ene, cyclohex-1,4-diene, cyclohex-1,5-diene, cyclohex-2,4-diene, and cyclohex-2,5-diene.

Examples of amantadine analogs that can be employed in the methods, compositions, and kits of the invention include the amantadine analogs selected from the group consisting of 1-amino-1,3,5-trimethylcyclohexane, 1-amino-1(trans),3(trans),5-trimethylcyclohexane, 1-amino-1(cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3(trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1(trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3(cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5-pentamethylcyclohexyl-1)-ethylamine semihydrate, N-(1,3,3,5,5-pentamethylcyclohexyl)-pyrrolidine, 1-amino-1,3(trans),5(trans)-trimethylcyclohexane, 1-amino-1,3(cis),5(cis)-trimethylcyclohexane, 1-amino-(1R,5S)trans-5-ethyl-1,3,3-trimethylcyclohexane, 1-amino-(1S,5S)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3(cis)-ethyl-cyclohexane, 1 -amino-1-methyl-3 (cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine or piperidine, N-[1,3(trans),5(trans)-trimethylcyclohexyl]pyrrolidine or piperidine, N-[1,3(cis),5(cis)-trimethylcyclohexyl]pyrrolidine or piperidine, N-(1,3,3,5-tetramethylcyclohexyl)pyrrolidine or piperidine, N-(1,3,3,5,5-pentamethylcyclohexyl)pyrrolidine or piperidine, N-(1,3,5,5-tetramethyl-3-ethylcyclohexyl)pyrrolidine or piperidine, N-(1,5,5-trimethyl-3,3-diethylcyclohexyl)pyrrolidine or piperidine, N-(1,3,3-trimethyl-cis-5-ethylcyclohexyl)pyrrolidine or piperidine, N-[(1S,5S)cis-5-ethyl-1,3,3-trimethylcyclohexyl]pyrrolidine or piperidine, N-(1,3,3-trimethyl-trans-5-ethylcyclohexyl)pyrrolidine or piperidine, N-[(1R,5S)trans-5-ethyl,3,3-trimethylcyclohexyl]pyrrolidine or piperidine, N-(1-ethyl-3,3,5,5-tetramethylyclohexyl)pyrrolidine or piperidine, N-(1-propyl-3,3,5,5-tetramethylcyclohexyl)pyrrolidine or piperidine, N-(1,3,3,5,5-pentamethylcyclohexyl)pyrrolidine, their optical isomers, diastereomers, enantiomers, hydrates, their pharmaceutically acceptable salts, and mixtures thereof. One amantadine analog is neramexane (1-amino-1,3,3,5,5-pentamethylcyclohexane), which is described, e.g., in U.S. Pat. No. 6,034,134.

Certain amantadine analogs of general formula (XIV) include the case where three axial alkyl substituent, e.g., Rₚ, Rᵣ and R₅ all together form a bridgehead to yield compounds (so called 1-aminoadamantanes) illustrated by the formulae XVb-XVd below:

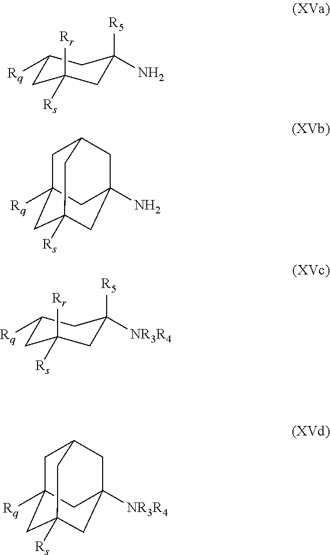

Certain amantadine analogs of formula (XIV) wherein n+m=0, U, V, W, X, Y and Z form a cyclohexane ring, and one or both of R₃ and R₄ are independently joined to the cyclohexane ring via alkylene bridges formed through Rₚ, R_{q}, Rᵣ, Rₛ or R₅ are represented by the following formulas XVIa-XVIC:

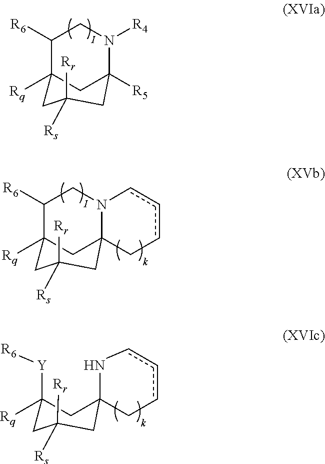

where R_{q}, Rᵣ, Rₛ, Rᵣ and R₅ are as defined above for formula (XIV), R₆ is hydrogen, linear or branched C₁-C₆ alkyl, linear or branched C₂-C₆ alkenyl, linear or branched C₂-C₆ alkynyl, aryl, substituted aryl or arylalkyl Y is saturated or may combine with R₆ to form a carbon-hydrogen bond with the ring carbon to which it is attached, l=0 or 1 and k=0, 1 or 2 and ______ represents a single or double bond.

Other amantadine analogs include 1-amino adamantane and its derivatives selected from the group consisting of 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5 -dihexyl adamantane, 1-amino-3-hexyl-5-cyclohexyl adamantane, 1-amino-3-hexyl-5-phenyl adamantane, 1-amino-3 -cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, 1-amino-3-butyl-5-cyclohexyl adamantane, their optical isomers, diastereomers, enantiomers, hydrates, N-methyl, N,N-dimethyl, N-ethyl, N-propyl derivatives, their pharmaceutically acceptable salts, and mixtures thereof.

The compounds of formulas XVb and XVd may be prepared by alkylation of halogenated adamantanes, preferably bromo- or chloroadamantanes. The di- or tri-substituted adamantanes may be obtained by additional halogenation and alkylation procedures. The amino group is introduced either by oxidation with chromiumtrioxide and bromination with HBr or bromination with bromine and reaction with formamide followed by hydrolysis. The amino function can be alkylated according to generally-accepted methods. Methylation can, for example, be effected by reaction with chloromethyl formate and subsequent reduction. The ethyl group can be introduced by reduction of the respective acetamide. For more details on synthesis see, e.g., U.S. Pat. Nos. 5,061,703 and 6,034,134.

Other amantadine analogs are described by formula XVII:

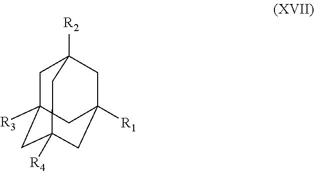

wherein R₁ is NHC(O)R₅, C(O)NHR₅, (CR₅R₆)ₙNR₅R₆ or (CR₅R₆)ₙCO₂R₅; n is an integer ranging from 0 to 4; R₂, R₃ and R₄ are each independently selected from the group consisting of H, fluoro, C₁-C₆ alkyl, and hydroxy; and each R₅ and R₆ is independently H or C₁-C₆ alkyl.

Amantadine analogs of formula XVII include methyl-3-fluoro-5-hydroxyadamantane-1-carboxylate; fluoroadamantane-1-carboxylic acid; 3,5-difluoro-adarnantan-1-ylamine; 3,5-difluoroadamantane-1-carboxylic acid; 3 -fluoroadamantan-1-ylamine; methyl-3,5-difluoro-7-hydroxyadamantane-1-carboxylate; 3,5,7-trifluoroadamantane-1-carboxylic acid; 3,5,7-trifluoroadamantan-1-ylamine; and the pharmaceutically acceptable salts of the foregoing compounds.

Still other amantadine analogs are described by formula XVIII:

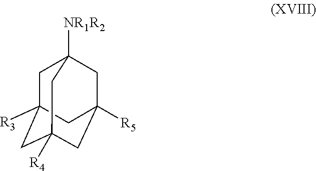

wherein each of R₁ and R₂ is independently hydrogen or a straight or branched C₁-C₆ alkyl or, in conjunction with N, a heterocyclic radical with 5 or 6 ring C atoms; each of R₃ and R₄ is independently hydrogen, a straight or branched C₁-C₆ alkyl, a C₅ or C₆ cycloalkyl, or phenyl; and R₅ is hydrogen or a straight or branched C₁-C₆ alkyl, or a pharmaceutically-acceptable acid addition salt thereof.

Amantadine analogs of formula XVIII include 1-amino adamantane, 1-amino-3-phenyl adamantane, 1-amino-methyl-adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, 1-amino-3 -butyl-5-cyclohexyl adamantane, their N-methyl, N,N-dimethyl, N-ethyl, N-propyl derivatives and their acid addition compounds.

Still other amantadine analogs are described by formula XIXa or formula XIXb.

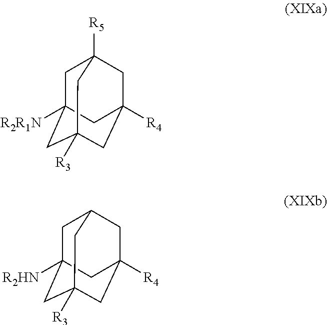

wherein R₁ is H, alkyl, heteroalkyl, aryl, heteroaryl, C(O)OR₆ or C(O)R₆; R₂ is H, alkyl, heteroalkyl, aryl, heteroaryl, C(O)OR₆, or C(O)R₆; R₃ is H, alkyl, heteroalkyl, aryl or heteroaryl; R₄ is H, alkyl, heteroalkyl, aryl or heteroaryl; R₅ is OR₇, alkyl-OR₇, or heteroalkyl-OR₇; R₆ is alkyl, heteroalkyl, aryl, or heteroaryl. R₇ is NO₂, C(O)R₆, C(O)alkyl-ONO₂, or C(O)heteroalkyl-ONO₂. The following substituents are preferred: R₁ and R₂ are H; R₃ and R₄ are H or alkyl; and R₇ is NO₂ or C(O)alkyl-ONO₂. Methods of making these compounds are described, for example, in U.S. Pat. No. 6,620,845.

Amantadine analogs of formula XIXa or XIXb include 1-acetamido-3,5-dimethyl-7-hydroxyadamantane, 1-amino-3,5-dimethyl-7-hydroxyadamantane hydrochloride, 1-tert-butylcarbamate-3,5-dimethyl-7-hydroxy-adamantane, 1-tert-butylcarbamate-3,5-dimethyl-7-nitrate-adamantane, 1-amino-3,5-dimethyl-7-nitrateadamantane hydrochloride, 1-acetamido-3,5-dimethyl-7-nitrateadamantane, 1,1-dibenzylamino-3,5-dimethyl-7-hydroxy-adamantane, 1-amino-3,5-dimethyl-7-acetoxyadamantane hydrochloride, 1-(benzyloxycarbonyl)amino-3,5-dimethyl-7-hydroxyadamantane, 1-(benzyloxycarbonyl)amino-3,5-dimethyl-7-(3-bromopropylcarbonyloxy)adamantane, 1-(benzyloxycarbonyl)amino-3,5-dimethyl-7-(3-nitratepropylcarbonyloxy)adamantane, 1-Acetamido-3,5-dimethyl-7-carboxylic acidadamantane, 1-acetamido-3,5-dimethyl-7-hydroxymethyladamantane, 1-amino-3,5-dimethyl-7-hydroxymethyladamantane hydrochloride, 1-(benzyloxycarbonyl)amino-3,5-dimethyl-7-hydroxymethyl adamantane, 1-(benzyloxycarbonyl)amino-3,5-dimethyl-7-nitratemethyl-adamantane, 1-amino-3,5-dimethyl-7-nitratemethyladamantane hydrobromide, and 1-acetamido-3,5-dimethyl-7-nitratemethyl-adamantane.

Amantadine analogs also include N-(1-adamantyl)diethylamine, N-(3-methyl-1-adamantyl)isopropylamine, N-(3,5-dimethyl-1-adamantyl)ethylmethylamine, N-(1-adamantyl)morpholine, N-(3,5,7-trimethly-1-adamantyl)piperidine, N,N′-bis(1-adamantyl)-1,3-propanediamine, N,N′-bis(3-methyl-1-adamantyl)-1,10-decanediamine, -bis(3,5,7-trimethyl-1-adamantyl)-1,6-hexanediamine, N-(1-adamantyl)cyclohexylamine, N-(1-adamantyl)cyclooctylamine, N-(1-adamantyl)-α-furfurylamine, N-(3-methyl-1-adamantyl)-β-thienylamine, N-(3,5,7-trimethyl-1-adamantyl)-α-furfurylamine, N-(1-adamantyl)-β-thienylamine, N-β-(2-pyridyl)ethyl-1-adamantylamine, N-(3,5-dimethyl-1-adamantyl)-5-phenylpentylamine, bis-adamantylamine, bis(3-methyl-1-adamantyl)amine, bis(3,5-dimethyl-1-adamantyl)amine, N-(1-adamantyl)dodecylamine, N-(1-adamantyl)-N′-phenylpiperazine, N-(1-adamantyl)piperazine, N-(1-adamantyl)aniline, N-(1-adamantyl)benzylamine, N-(1-adamantyl)phenethylamine, N-(1-adamantyl)homoveratylamine, bis(3,5,7-trimethyl-1-adamantyl)amine, N-(3,5,7-trimethyl-1-adamantyl)-1-adamantylamine, 1-amino adamantane, and N-(3,5,7-trimethyl-1-adamantyl)-N′-phenylpiperazine.

Amantadine analogs also include adatanserin, tromantadine, amantanium bromide, rimantadine, somantadine, adapalene, N-1-adamantyl-N′-cyclohexyl-4-morpholinecarboxamidine, dopamantine, adaprolol maleate, (−)-N-(2-(8-methyl-1,4-benzodioxan-2-ylmethylamino)ethyl)adamantane-1-carboxamide, N-(1-adamantyl)-N′,N′-(1,5-(3-(4(5)-1H-imidazolyl-pentanediyl)))formamidine, adamantoyl-Lys-Pro-Tyr-Ile-Leu, 1-(2-pyridyl)-4-(1-methyl-2-(1-adamantylamino) ethyl)piperazine, adafenoxate, (1R,3S)-3-(1-adamantyl)-1-aminomethyl-3,4-dihydro-5,6-dihydroxy-1H-2-benzopyran, adamantylamide L-Ala-L-isoGlu, 2-adamantylamino-benzoic acid, N(alpha)-(1-adamantanesulphonyl)-N-(4-carboxybenzoyl)-L-lysyl-alanyl-L-valinal, 4-acylamino-1-aza-adamantane, L-leucyl-D-methionyl-glucyl-N-(2-adamantyl)-L-phenylal anyl amide, Tyr-(D)-Met-Gly-Phe-adamantane, 1-N-(p-bromobenzoyl)methyladamantylamine, 4-butyl-1,2-dihydro-5-((1-adamantanecarbonyl)oxy)-1,2-diphenyl-3H-pyrazol-3-one, N(alpha)-(1-adamantanesulphonyl)-N(epsilon)-succinyl-L-lysyl-L-prolyl-L-valinal, and the amantadine salt of N-acetyl-DL-phenylalanine.

Amantadine analogs also include (2-hydroxy-adamantan-2-yl)-acetic acid ethyl ester, (2-methyl-adamantan-2-yloxy)-acetic acid, (2-piperidin-1-yl-adamantan-2-yl)-methylamine, (4-adamantan-1-yl)-thiazol-2-ylamine, (4-adamantan-1-yl-phenoxy)-acetic acid (4-tricyclo[3.3.1.13,7]decan-1-yl-phenoxy-acetic acid), (adamantan-1-ylmethoxy)-acetic acid, (adamantan-1-yloxy)-acetic acid, (adamantan-1-ylsulfanyl)-acetic acid, (tricyclo[3.3.1.13,7]decan-1-carbonyl-3-aminophenyl-amide), [3-(3,4-dimethyl-phenyl)-adamantan-1-yl]-methylamine, 1-(1-adamantypethyl (2-nitro-5-piperazinophenypamine, 1-(1-adamantyl)ethyl(5-chloro -2-nitrophenyl)amine, 1-(1-adamantyl)ethylamine hydrochloride, 1-(4-hexahydro-1-pyrazinyl-3-nitrophenylcarboxamido)-3,5-dimethyladamantane, 1-(4-hexahydro-1-pyrazinyl-3-nitrophenylcarboxamido)-adamantane, 1,3-adamantanediacetic acid, 1,3-adamantanedicarboxamide, 1,3-adamantanedicarboxylic acid, 1,3-adamantanedimethanol, 1,3-dibromoadamantane, 1,3-dihydroxyadamantane (1,3-adamantanediol), 1,3-dimethyladamantane, 1,4-dibromoadamantane, 1-[1-(4-hexahydro-1-pyrazinyl-3-nitrophenylcarboxamido)-ethyl]adamantane, 1-acetamidoadamantane, 1-adamantan-1-yl-2-methyl-propan-1-one, 1-adamantan-1-yl-2-phenyl-ethanone, 1-adamantan-1-yl-3-methyl-butan-1-one, 1-adamantan-1-yl-3-phenyl-propan-1-one, 1-adamantan-1-yl-butan-1-one, 1-adamantan-1-yl-butan-2-one, 1-adamantan-1-yl-propan-1-one, 1-adamantan-1-yl-propan-2-one, 1-adamantanamine, 1-adamantanamine hydrochloride, 1-adamantanamine sulfate, 1-adamantaneacetic acid, 1-adamantaneacetyl chloride, 1-adamantanecarbonitrile, 1-adamantanecarbonyl chloride, 1-adamantanecarboxamide, 1-adamantanecarboxylic acid, 1-adamantaneethanol, 1-adamantanemethanol, 1-adamantanemethylamine, 1-adamantanol (1-hydroxyadamantane), 1-adamantyl bromomethyl ketone, 1-adamantyl methyl ketone, 1-amino-3-hydroxy-adamantane hydrochloride, 1-aminoadamantane sulfate(bis[1-aminotricyclo(3.3.1.1.3.7)decane]sulfate), 1-bromo-3,5-dimethyladamantane, 1-bromoadamantane, 1-chloro-3,5-dimethyladamantane, 1-chloroadamantane, 1-hydroxy-3,5-dimethyladamantane, 1-hydroxy-3-amino-5,7-dimethyladamantane hydrochloride, 1-hydroxy-3-nitro -5,7-dimethyladamantane, 1-isocyanato-adamantane (1-isocyanato-tricyclo[3.3.1.13,7]decane), 1-nitro-3,5-dimethyladamantane, 2-(1-adamantyl)-4,5-dichloropyridazin-3(2H)-one (4,5-dichloro-2-tricyclo[3.3.1.13,7]decan-1-yl-2h-pyridazin-3-one), 2-(1-adamantyl)-5-(chloromethyl)-1,3-thiazole (5-chloromethyl-2-tricyclo[3.3.1.13,7]decan-1-yl-thiazole), 2-(4-hexahydro-1-pyrazinyl-3-nitrophenylcarboxamido)-adamantane, 2-(adamantan-1-ylamino)-ethanol (2-(tricyclo[3.3.1.13,7]decan-1-ylamino)-ethanol), 2-(adamantan-1-ylthio)-ethanamine (2-(tricyclo[3.3.1.13,7]decan-1-ylsulfanyl)-ethylamine), 2-(adamantan-2-ylamino)-ethanol, 2-[(adamanatn-1-ylmethyl)-amino]-ethanol hydrochloride, 2-adamantan-1-yl-ethylamine, 2-adamantanamine hydrochloride, 2-adamantanol, 2-adamantanone (2-hydroxyadamantane), 2-adamantanone oxime, 2-aminoadamantane hydrochloride (2-adamantanamine HCl), 2-bromoadamantane, 2-ethyl-2-adamantanol, 2-methyl-2-adamantanol, 2-methyl-2-adamantyl acrylate, 2-piperidin-1-yl-adamantane-2-carbonitrile, 3-(3,4-dimethyl-phenyl)-adamantane-1-carboxylic acid, 3-(adamantan-1-yl)-3-oxo-propionitrile, 3-(adamantan-1-yl)-4-hydroxy-5-methoxy-benzoic acid, 3-(adamantan-1-ylsulfanyl)-[1,2,4]-thiadiazol-5-ylamine (3-(tricyclo[3.3.1.13,7]decan-1-ylsulfanyl)-1,2,4-thiadiazol-5-ylamine), 3-(adamantan-1-ylsulfanyl)-propylamine, 3,5-dimethyl-1-adamantanol, 3-adamantan-1-yl-3-oxo-propionic acid ethyl ester (tricyclo[3.3.1.13,7]decane-1-propanoic acid, β-oxo-ethyl ester), 3-adamantan-1-yl-4-methoxy-benzoic acid (4-methoxy-3-tricyclo[3.3.1.13,7]decan-1-yl-benzoic acid), 3-hydroxyadamantane-1-carboxylic acid, 3-noradamantanecarboxylic acid, 4,4′-(1,3-adamantanediyl)diphenol, 4-adamantan-1-yl-1,2,3-thiadiazole (4-tricyclo[3.3.1.13,7]dec-1-yl-1,2,3-thiadiazole), 4-adamantan-1-yl-2-aminophenol (2-amino-4-tricyclo[3.3.1.13,7]decan-1-yl-phenol), 4-adamantan-1-yl-5-ethyl-thiazol-2-ylamine, 4-adamantan-1-yl-5-isopropyl-thiazol-2-ylamine, 4-adamantan-1-yl-5-methyl-thiazol-2-ylamine, 4-adamantan-1-yl-5-phenyl-thiazol-2-ylamine, 4-aza-tricyclo[4.3.1.13,8]undecan-5-one, 4-aza-tricyclo[4.3.1.13,8]undecane, 5′-methylspiro [adamantan-2,2′-[1,3]-dioxane]5′-carboxylic acid 5′-methylspiro[adamantan-2,2′-[1,3]-dioxane]-5′-amine, 5-adamantan-1-yl-[1,3,4]-oxadiazole-2-thiol(2-thiol-5-tricyclo[3.3.1.13,7]dec-1-yl-1,3,4-oxadizol), 5-adamantan-1-yl-2h-pyrazole-3-carboxylic acid methyl ester, 5-adamantan-1-yl-2-methoxy-benzoic acid (2-methoxy-5-tricyclo[3.3.1.13,7]decan-1-yl-benzoic acid), 5-adamantan-1-yl-2-methyl-furan-3-carboxylic acid (5-tricyclo[3.3.1.13,7]decan-1-yl-furan-3-carboxylic acid), 5-adamantan-1-yl-2-methyl-furan-3-carboxylic acid methyl ester (5-tricyclo[3.3.1.13,7]decan-1-yl-furan-3-carboxylic acid methyl ester), 5-adamantan-1-yl-2-methyl-phenylamine (2-methyl-5-tricyclo[3.3.1.13,7]decan-1-yl-phenylamine), 5-adamantan-1-yl-3-ethyl-isoxazole-4-carboxylic acid, 5-adamantan-1-yl-3-methyl-isoxazole-4-carboxylic acid, 5-adamantan-1-yl-furan-2-carboxylic acid (5-tricyclo[3.3.1.13,7]decan-1-yl-furan-2-carboxylic acid), 5-adamantan-1yl-furan-2-carboxylic acid methyl ester (5-tricyclo[3.3.1.13,7]decan-l-yl-furan-2-carboxylic acid methyl ester), 5-chloro-2-nitrophenyl(adamantan-2-yl)amine, 5-hydroxy-2-adamantanone, adamantan-1-yl-methylamine, adamantan-2-ylidene-acetonitrile, adamantane, adamantane-1-carbonyl isothiocyanate (tricyclo[3.3.1.13,7]decane-1-carbonyl isothiocyanate), adamantane-1-carbothioic acid amide (tricyclo[3.3.1.13,7]decane-1-carbothioic acid amide), adamantane-1-carboxylic acid (3-amino-phenyl)-amide, adamantane-1-carboxylic acid (4-amino-2-methoxy-phenyl)-amide (tricyclo[3.3.1.13,7]decan-1-carbonyl-2-methoxy-3-aminophenyl-amide), adamantane-1-carboxylic acid (4-amino-phenyl)-amide (tricyclo[3.3.1.13,7]decan-1-carbonyl-4-aminophenyl-amide), adamantane-1-sulfinyl chloride, congressane, dimethyl 1,3-adamantanedicarboxylate, dimethyl-1,3-adamantanedicarboxylate, ethyl 1-adamantanecarboxylate, methyl 1-adamantanecarboxylate, N-(1-adamantyl)ethylenediamine, N-(1-adamantyl)urea, N-(2-adamantyl)-N-(4-bromophenyl)amine, N-(adamantan-2-yl)-N-(2-chloro-ethyl)-amine hydrochloride, N-2-(5-hexahydro-1-pyrazinyl-2-nitrophenyl)adamantan-2-yl-amine, N-adamantan-1-oyl-piperazine, N-adamantan-1-yl-2-amino-benzamide (2-amino-N-tricyclo[3.3.1.13,7]decan-1-yl-benzamide), N-formyl-1-amino-3,5-dimethyladamantane, N-methyl-(adamantan-1-yl)methylamine, and p-(1-adamantyl)phenol.

### T-705

T-705 (6-fluoro-3-hydroxy-2-pyrazinecarboxamide) is an inhibitor of viral polymerase and has been found to have potent inhibitory activity against influenza A, B, and C. Studies have suggested that host cell kinases convert T-705 into the active form T-705 ribofuranosyl triphosphate (T-705 RTP), which inhibits viral polymerase without affecting host cellular RNA or DNA synthesis. T-705 can be administered orally. The structure of T-705 is given below:

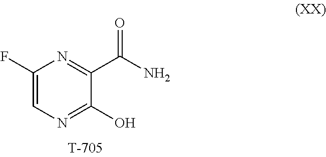

### Additional PDE Inhibitors

Other PDE 4 inhibitors are described in the following patents, patent applications, and references: U.S. Pat. Nos., 6,656,717, 6,699,890, 7,087,625, 7,153,871, 7,205,320, 7,226,930, 7,226,930, 7,235,579, 7,332,486, 7,335,654, 7,342,021, 7,396,833, 7,405,230, 7,429,664, 7,432,266, 7,488,737, 7,495,017, 7,576,080, 7,585,882, 7,625,924, 7,693,831, 7,655,802, 7,696,198, 7,696,229; U.S. Patent Publication Nos. 20020151566, 20030045533, 20030139406, 20030149052, 20040087584, 20040102460, 20050119225, 20050289660, 20060166316, 20060168668, 20070093510, 20080039462, 20080200471, 20090054434, 20090069337, 20090069337, 20100010017, 20100016297, 20100022581, 20100029629, 20100029689, 20100056491, 20100056531; International Publication Nos. WO 01/00851, WO 02/074726, WO 02/098878, WO 03/032981, WO 03/044170, WO 03/091694, WO 04/009552, WO 04/009557, WO 04/014911, WO 04/014913, WO 04/019943, WO 04/029050, WO 04/046113, WO 04/089285, WO 04/090126, WO 04/094375, WO 04/094411, WO 05/0051389, WO 05/061458, WO 05/063767, WO 05/092890, WO 05/111038, WO 06/001894, WO 06/028957, WO 06/044528, WO 06/044955, WO 06/069097, WO 06/071988, WO 06/135828, WO 07/022280, WO 07/038367, WO 07/056582, WO 07/098169, WO 07/098214, WO 07/098418, WO 07/100880, WO 07/103260, WO 07/103370, WO 07/103554, WO 07/123953, WO 08/101247, WO 08/147812, WO 09/023844, WO 09/029214, WO 09/055437, WO 09/067607, WO 10/002802, WO 10/003084, WO 10/008832, WO 10/01546, WO 10/021797, WO 10/0024980; and European Patent Nos. EP 1 249 619, EP 1 235 933, EP 1 529 049, EP 1 249 619, EP 1 697 378, EP 1 828 179; German Patent Pub. No. DE 102007 022945 and Burgin, Alex B. et al. Nature Biotechnology, 25:1 (2010), each of which is incorporated herein by reference.

### Combinations

The invention includes the individual combination of each neuraminidase with each PDE inhibitor provided herein and, optionally, amantadine, rimantadine, or T-705, as if each combination were explicitly stated. In a particular example, the antiviral agent is oseltamivir, zanamivir, or peramivir, and the PDE inhibitor is ibudilast, roflumilast, or rolipram. In another example, the combination comprises oseltamivir, ibudilast, and amantadine. In still another example, the antiviral agent is oseltamivir, zanamivir, or peramivir, and the PDE inhibitor is DG-071, D 157140, D 158681, D 159382, D159404, D 159687, MEM1414, MEM 1917 or an analog thereof.

### Delivery of Compound(s)

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Administration

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. In one example, RPL554 is administered intranasally.

In particular embodiments of any of the methods of the invention, multiple compounds are administered within 28 days of each other, within 14 days of each other, within 10 days of each other, within five days of each other, within twenty-four hours of each other, or simultaneously. Combinations of compounds may be formulated together as a single composition, or may be formulated and administered separately. Each compound may be administered in a low dosage or in a high dosage, each of which is defined herein.

In combination therapy, the dosage and frequency of administration of each component of the combination can be controlled independently. For example, one compound may be administered three times per day, while a second compound may be administered once per day. Combination therapy may be given in on-and-off cycles that include rest periods so that the patient's body has a chance to recover from any as yet unforeseen side effects. The compounds may also be formulated together such that one administration delivers both compounds.

### Formulation of Pharmaceutical Compositions

The administration of a combination of the invention may be by any suitable means that results in suppression of proliferation at the target region. A compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

The pharmaceutical formulation can be a sustained release oral pharmaceutical formulation which provides for controlled, modified, delayed and/or sustained release of the anti-influenza viral agent. Such formulations can be prepared by methods known in the art. The pharmaceutical formulations are useful for administering anti-influenza viral agent to mammals including, but not limited to, horses, rodents, cows, pigs, dogs, cats, primates, chickens, birds, fowl and particularly human

Each compound in a combination may be formulated in a variety of ways that are known in the art. For example, all agents may be formulated together or separately. Desirably, all agents are formulated together for the simultaneous or near simultaneous administration of the agents. Such co-formulated compositions can include all compounds formulated together in the same pill, capsule, liquid, etc. It is to be understood that, when referring to the formulation of particular combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Stabilizing additives may be incorporated into the composition. With some drugs, the presence of such additives promotes the stability and dispersibility of the agent in solution. The stabilizing additives may be employed at a concentration ranging from about 0.1 and 5% (W/V), preferably about 0.5% (WN). Suitable, but non-limiting, examples of stabilizing additives include gum acacia, gelatin, methyl cellulose, polyethylene glycol, carboxylic acids and salts thereof, and polylysine. The preferred stabilizing additives are gum acacia, gelatin and methyl cellulose.

The oral dosage forms of the present invention, containing a mixture of one or more active agents, e.g., a PDE4 Inhibitor and a neuraminidase inhibitor and may include additional materials known to those skilled in the art as pharmaceutical excipients. Any excipient or ingredient, including pharmaceutical ingredients or excipients. Such pharmaceutical excipients include, for example, the following: Acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); Aerosol propellants (butane, dichlorodifluoro-methane, dichlorotetrafluoroethane, isobutane, propane, trichloromonofluoromethane); Non-Fluorocarbon-containing propellants (hydrofluoroalkane (HFA)); Air displacements (carbon dioxide, nitrogen); Alcohol denaturants (denatonium benzoate, methyl isobutyl ketone, sucrose octacetate); Alkalizing agents (strong ammonia solution, ammonium carbonate, diethanolamine, diisopropanolamine, potassium hydroxide, sodium bicarbonate, sodium borate, sodium carbonate, sodium hydroxide, trolamine); Anticaking agents (see glidant); Antifoaming agents (dimethicone, simethicone); Antimicrobial preservatives (benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Antioxidants (ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); Buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); Capsule lubricants (see tablet and capsule lubricant); Chelating agents (edetate disodium, ethylenediaminetetraacetic acid and salts, edetic acid); Coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein); Colorants (caramel, red, yellow, black or blends, ferric oxide); Complexing agents (ethylenediaminetetraacetic acid and salts (EDTA), edetic acid, gentisic acid ethanolmaide, oxyquinoline sulfate); Desiccants (calcium chloride, calcium sulfate, silicon dioxide); Emulsifying and/or solubilizing agents (acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax); Filtering aids (powdered cellulose, purified siliceous earth); Flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Glidants and/or anticaking agents (calcium silicate, magnesium silicate, colloidal silicon dioxide, talc); Humectants (glycerin, hexylene glycol, propylene glycol, sorbitol); Plasticizers (castor oil, diacetylated monoglycerides, diethyl phthalate, glycerin, mono- and di-acetylated monoglycerides, polyethylene glycol, propylene glycol, triacetin, triethyl citrate); Polymers (e.g., cellulose acetate, alkyl celloloses, hydroxyalkylcelloloses, acrylic polymers and copolymers); Solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water); Sorbents (powdered cellulose, charcoal, purified siliceous earth); Carbon dioxide sorbents (barium hydroxide lime, soda lime); Stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); Suspending and/or viscosity-increasing agents (acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); Tablet binders (acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, syrup); Tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner=s sugar); Tablet disintegrants (alginic acid, microcrystalline cellulose, croscarmellose sodium, corspovidone, polacrilin potassium, sodium starch glycolate, starch, pregelatinized starch); Tablet and/or capsule lubricants (calcium stearate, glyceryl behenate, magnesium stearate, light mineral oil, polyethylene glycol, sodium stearyl fumarate, stearic acid, purified stearic acid, talc, hydrogenated vegetable oil, zinc stearate); Tonicity agent (dextrose, glycerin, mannitol, potassium chloride, sodium chloride); Vehicle: flavored and/or sweetened (aromatic elixir, compound benzaldehyde elixir, iso-alcoholic elixir, peppermint water, sorbitol solution, syrup, tolu balsam syrup); Vehicle: oleaginous (almond oil, corn oil, cottonseed oil, ethyl oleate, isopropyl myristate, isopropyl palmitate, mineral oil, light mineral oil, myristyl alcohol, octyldodecanol, olive oil, peanut oil, persic oil, seame oil, soybean oil, squalane); Vehicle: solid carrier (sugar spheres); Vehicle: sterile (bacteriostatic water for injection, bacteriostatic sodium chloride injection); Viscosity-increasing (see suspending agent); Water repelling agent (cyclomethicone, dimethicone, simethicone); and Wetting and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol). This list is not meant to be exclusive, but instead merely representative of the classes of excipients and the particular excipients which may be used in oral dosage forms of the present invention.

### Dosages

The dosage of a compound or a combination of compounds depends on several factors, including: the administration method, the type of viral infection to be treated, the severity of the infection, whether dosage is designed to treat or prevent a viral infection, and the age, weight, and health of the patient to be treated.

For combinations identified herein, the recommended dosage for the anti-viral agent is can be less than or equal to the recommended dose as given in the Physician's Desk Reference, 60^{th} Edition (2006). In other cases, the dosage of the compound or antiviral agent may be higher than the recommended dose.

Combinations of the following are preferred embodiments of the invention: oseltamivir and rolipram, oseltamivir and ibudilast, oseltamivir and roflumilast, oseltamivir and DG-071, oseltamivir and D 157140, oseltamivir and D 158681, oseltamivir and D 159382, oseltamivir and D159404, oseltamivir and D 159687, oseltamivir and MEM1414 and oseltamivir and MEM 1917.

Another embodiment of the invention provides a pharmaceutical composition containing from about 1800 mg to about 3600 mg of peramivir and an effective amount of one or more PDE4I.

Yet another embodiment of the invention comprises an oral pharmaceutical composition comprising an effective amount of peramivir and an effective amount of one or more PDE4I.

Another embodiment of the invention provides a pharmaceutical composition containing from about 10 mg to about 1000 mg of zanamivir and an effective amount of one or more PDE4I.

Yet another embodiment of the invention provides a pharmaceutical composition comprising an effective amount of zanamivir and an effective amount of one or more PDE4I.

Yet another embodiment provides a pharmaceutical composition comprising zanamivir and one or more PDE4I where the mean Cmax of zanamivir is from about 17 ug/mL to about 142 ug/mL when administered to a human. In another embodiment, the AUC for oral administration of zanamivir is such that it is considered a bioequivalent to inhaled forms of zanamivir.

Yet another embodiment of the invention provides a pharmaceutical composition comprising zanamivir and one or more PDE4I where the median Tmax is from about 0.5 hours to about 3 hours when administered to a mammal, e.g., a human.

Yet another embodiment of the invention provides a pharmaceutical composition comprising zanamivir and one or more PDE4I where the median Tmax is from about 1 hour to about 2 hours when administered to a mammal, e.g., a human.

Yet another embodiment of the invention provides a pharmaceutical composition comprising zanamivir and one or more PDE4I where the AUC is about 80 ng/hr/mL to about 500 ng/hr/mL when administered orally to a mammal, e.g. a human.

Yet another embodiment of the invention is a pharmaceutical composition comprising zanamivir and one or more PDE4I where the AUC is about 111 ng/hr/mL to about 164 ng/hr/mL when administered orally to a mammal, e.g. a human.

Yet another embodiment of the invention is a pharmaceutical composition comprising zanamivir and one or more PDE4I where the half life is about 2.5 hours to about 5.1 hours in a mammal, e.g. a human.

Yet another embodiment of the present invention provides a pharmaceutical composition that comprises one or more PDE4I and a sufficient amount of zanamivir such that the pharmaceutical composition achieves a maximum plasma concentration of zanamivir of about 10 ng/ml to about 200 ng/ml in about 1 to 2 hours in a mammal, e.g. a human, when administered orally by a mammal, e.g., a human.

Yet another embodiment of the present invention provides a pharmaceutical composition that comprises one or more PDE4I and a sufficient amount of zanamivir such that the pharmaceutical composition achieves an Area Under the Curve of about 10 ng/hr/mL to about 2000 ng/hr/mL when administered orally to a mammal, e.g. a human.

Yet another embodiment of the present invention provides a pharmaceutical composition that comprises one or more PDE4I and about 0.01 mg/kg to about 100 mg/kg of oseltamivir.

Yet another embodiment of the present invention provides a pharmaceutical composition that comprises one or more PDE4I and a sufficient amount of BCX-1898 to treat or prevent influenza virus infections in a mammal, e.g. a human.

Yet another embodiment of the present invention provides a pharmaceutical composition that comprises one or more one or more PDE4I and a sufficient concentration of BCX-1923 to treat or prevent influenza virus infections in a mammal, e.g. a human.

Yet another embodiment of the present invention provides a pharmaceutical composition that comprises one or more PDE4I and a sufficient concentration of BCX-1827, BCX-1989 or BCX 1827 to treat or prevent influenza virus infections in a mammal, e.g. a human.

As described above, the compound in question may be administered orally in the form of tablets, capsules, elixirs or syrups, rectally in the form of suppositories or via inhalation route. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. The correct dosage of a compound can be determined by examining the efficacy of the compound in viral replication assays, as well as its toxicity in humans.

An agent is usually given by the same route of administration that is known to be effective for delivering it as a monotherapy. For example, when used in combination therapy an agent is dosed in amounts and frequencies equivalent to or less than those that result in its effective monotherapeutic use.

A combination described herein may be administered to the patient in a single dose or in multiple doses. Components of the combination may be administered separately or together, and by the same or different routes. In addition, various components of the combination may be administered at the same or different times. When multiple doses are administered, the doses may be separated from one another by, for example, one, two, three, four, or five days; one or two weeks; or one month. For example, the combination may be administered once a week for, e.g., 2, 3, 4, 5, 6, 7, 8, 10, 15, 20, or more weeks. Both the frequency of dosing and length of treatment may be different for each compound of the combination. It is to be understood that, for any particular subject, specific dosage regimes should be adjusted over time according to the individual need and the professional judgment of the person administering or supervising the administration of the compositions. For example, the dosage of the combination, or components thereof, can be increased if the lower dose does not sufficiently treat the viral infection. Conversely, the dosage of the combination can be decreased if the viral infection is cleared from the patient.

In other embodiments, agents, either as monotherapies in combination with other agents can be administered at higher dosages than the recommended dosage.

### EXAMPLE 1

### In Vivo Activity of Compositions Comprising Oseltamivir and a PDE Inhibitor in an Influenza Mouse Model

### Virus

Mouse-adapted influenza A/NWS/33 (H1N1), which was not oseltamivir-resistant, was procured from the American Type Culture Collection (ATCC) at a virus titer of 10^{7.19} CEID₅₀/mL. The virus stock was diluted in phosphate buffered saline (PBS) to a working concentration of 10⁴⁵ TCID₅₀ of virus per 50 μL.

### Animals

Specific-pathogen-free, male C57/BL6 mice weighing 20-25 g were procured from Biological Resource Centre (BRC) and housed in groups of five in cages with Corncob bedding (Harlan-Teklad, U.K.). Experiments were conducted in Animal Bio-safety level 3 (ABSL-3) rooms. Cages were placed in isolators maintained at −100 Pa pressure and supplied with HEPA filtered air. Mice were provided with a commercial rodent diet (Harlan-Teklad, U.K.) and distilled water ad libitum.

### Procedure

Individual mice were anesthetized with ketamine (75 mg/kg) and xylazine (50 mg/kg) and intranasally administered with 50 μL of 10⁴⁵ TCID₅₀ virus suspension. In earlier experiments, we observed that a viral load of 10^{4.5} TCID₅₀/mouse is approximately five times the MLD₅₀ and produces 100% mortality in C57/BL6 mice (data not shown). Rolipram, ibudilast and roflumilast were suspended in 0.5% HPMC while oseltamivir was dissolved in distilled water. Starting twenty-four hours after virus inoculation, mice were orally administered with respective treatments twice daily for 5 days. Mice were weighed daily and the weights were used for dose adjustments. Animal survival was monitored for 20 days.

### Results

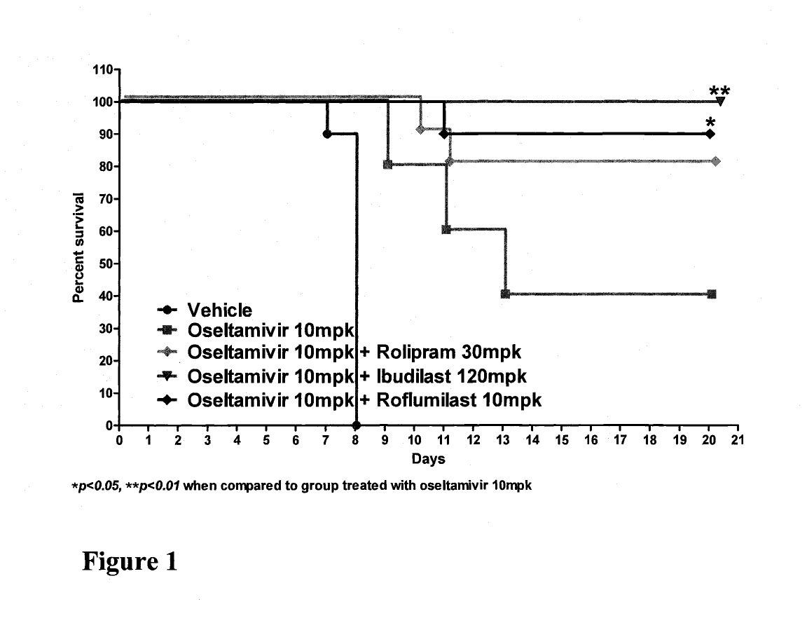

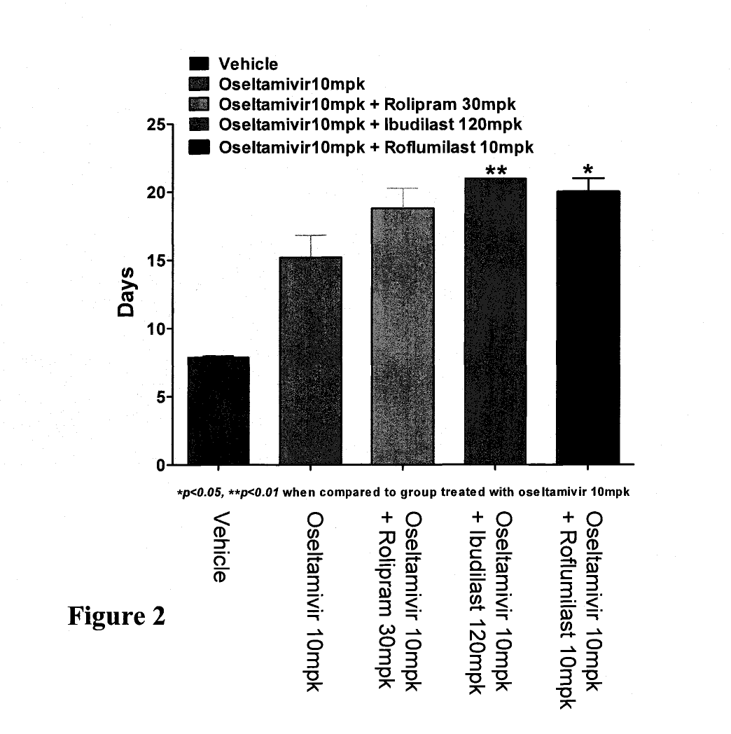

Vehicle treated mice began to die on day 7 and their survival rate on day 8 was 0%. Treatment with rolipram, ibudilast, or roflumilast alone also gave 0% survival on day 8. The survival rate for mice treated with oseltamivir alone at 10 mg/kg/day was 40%. Mice treated with a combination of oseltamivir at 10 mg/kg/day and a PDE4 inhibitor showed increased survival and mean day to death. Mice treated with the combinations oseltamivir and rolipram, oseltamivir and ibudilast, and oseltamivir and roflumilast had 80%, 100% and 90% survival rates, respectively (FIGS. 1 and 2).

These results demonstrate that a PDE inhibitor enhances the efficacy of a co-administered antiviral compound against influenza in an in vivo model. Thus, PDE inhibitors are useful as preventive and therapeutic agents against influenza in combination with antiviral agents such as neuraminidases.

### Other Embodiments

All patents, patent applications, and publications mentioned in this specification are herein incorporated by reference to the same extent as if each independent patent, patent application, or publication was specifically and individually indicated to be incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of molecular biology, medicine, immunology, pharmacology, virology, or related fields are intended to be within the scope of the invention.

What is claimed is:

1. A composition comprising:
(a) a first compound that is a neuraminidase inhibitor; and
(b) a second compound that is a PDE inhibitor.

2. The composition of claim 1, wherein said neuraminidase inhibitor is oseltamivir, zanamivir, peramivir, or an analog thereof.

3. The composition of claim 1, wherein said PDE inhibitor is a compound in Table 1 or an analog thereof.

4. The composition of claim 3, wherein said PDE inhibitor is ibudilast, rolipram, roflumilast, or an analog thereof.

5. The composition of claim 1, further comprising a third compound that is amantadine, rimantadine, T-705, or an analog thereof.

6. The composition of claim 1, wherein said first compound and said second compound are present in amounts that together are effective to treat or prevent a viral infection caused by an influenza virus.

7. The composition of claim 6, wherein said influenza virus is a type A influenza virus.

8. The composition of claim 6, wherein said influenza virus is a type B influenza virus.

9. The composition of claim 6, wherein said influenza virus is a type C influenza virus.

10. The composition of claim 7, wherein said influenza virus is a subtype H1N1 influenza virus.

11. The composition of claim 6, wherein said influenza virus is oseltamivir resistant.

12. The composition of claim 6, wherein said influenza virus is not oseltamivir resistant.

13. The composition of claim 1, wherein said composition is formulated for oral or systemic administration.

14. The composition of claim 1, wherein said composition consists of one or more pharmaceutically acceptable excipients and active ingredients, wherein said active ingredients consist of said first compound and said second compound.

15. The composition of claim 5, wherein said composition consists of one or more pharmaceutically acceptable excipients and active ingredients, wherein said active ingredients consist of said first compound, said second compound, and said third compound.

16. A method for treating or preventing a viral infection caused by an influenza virus in a patient, said method comprising administering to said patient a first compound that is a neuraminidase inhibitor and a second compound that is a PDE inhibitor in amounts that together are sufficient to treat or prevent said viral infection in said patient.

17. The method of claim 16, wherein said neuraminidase inhibitor is oseltamivir, zanamivir, peramivir, or an analog thereof.

18. The method of claim 16, wherein said PDE inhibitor is a compound in Table 1 or an analog thereof.

19. The method of claim 16, wherein said PDE inhibitor is ibudilast, rolipram, roflumilast, or an analog thereof.

20. The method of claim 16, further comprising administering to said patient a third compound that is amantadine, rimantadine, T-705, or an analog thereof.

21. The method of claim 16, wherein said influenza virus is a type A influenza virus.

22. The method of claim 16, wherein said influenza virus is a type B influenza virus.

23. The method of claim 16, wherein said influenza virus is a type C influenza virus.

24. The method of claim 16, wherein said influenza virus is a subtype H1N1 influenza virus.

25. The method of claim 16, wherein said influenza virus is oseltamivir resistant.

26. The method of claim 16, wherein said influenza virus is not oseltamivir resistant.

27. The method of claim 16, wherein said administration is oral or systemic.

28. The method of claim 16, wherein said first compound and said second compound are administered within 7 days of each other.

29. The method of claim 28, wherein said first compound and said second compound are administered within 1 day of each other.

30. The method of claim 28, wherein said first compound and said second compound are administered within 1 hour of each other.

31. The method of claim 28, wherein said first compound and said second compound are administered substantially simultaneously.

32. A kit comprising:
(a) a neuraminidase inhibitor; and
(b) a PDE inhibitor; and
(c) instructions for administering (a) and (b) to a patient for treating or preventing an influenza viral infection.

33. A kit comprising:
(a) a neuraminidase inhibitor; and
(b) instructions for administering (a) with at least one PDE4 inhibitor to a patient for treating or preventing a viral infection caused by influenza virus.

34. A kit comprising:
(a) a PDE inhibitor; and
(b) instructions for administering (a) with at least one neuraminidase inhibitor to a patient for treating or preventing a viral infection caused by influenza virus.

35. A kit comprising:
(a) a neuraminidase inhibitor; and
(b) a PDE inhibitor; and
(c) amantadine, rimantadine, or T-705; and
(d) instructions for administering (a), (b), and (c) to a patient for treating or preventing a viral infection caused by influenza virus.

36. A kit comprising:
(a) a neuraminidase inhibitor; and
(b) a PDE inhibitor; and
(c) instructions for administering (a) and (b) with amantadine, rimantadine, or T-705 to a patient for treating or preventing a viral infection caused by influenza virus.
